(19) United States
(12) Patent Application Publication
(10) Pub. No.: US 2025/0087358 A1
(43) Pub. Date: Mar. 13, 2025

(54) MEDICAL SUPPORT SYSTEM

(71) Applicant: ALLM INC., Tokyo (JP)

(72) Inventors: Teppei SAKANO, Tokyo (JP); Hiroyuki TAKAO, Tokyo (JP); Nogueira Raul Gomes, Tokyo (JP); Kayoko TAKAO, Nagasaki (JP)

(21) Appl. No.: 18/292,556

(22) PCT Filed: Aug. 12, 2021
(86) PCT No.: PCT/JP2021/029756

§ 371 Date: Jan. 26, 2024

(51) Int. Cl. G16H 50/30; G16H 10/20; G16H 40/67; H04W 4/02

(52) U.S. Cl. CPC G16H 50/30; G16H 10/20; G16H 40/67; H04W 4/025

(57) ABSTRACT

A medical support system includes: a vital data acquisition means configured to acquire vital data including blood pressure data, pulse data, electrocardiogram data, and oxygen saturation data of a subject; an abnormality detection means configured to, upon determining that there is a possibility that the subject has developed heart disease, brain disease, or an infectious disease on the basis of the vital data detect an abnormality in the subject; a determination means configured to, upon the abnormality detection means detecting the abnormality, determine the severity of symptoms of the subject; a transport destination specification means configured to specify a transport destination hospital to which the subject is transported, on the basis of the severity of the symptoms of the subject; and a transport destination information output means configured to output transport destination information about the transport destination hospital.

### TECHNICAL FIELD

The present invention relates to a medical support system.

### BACKGROUND ART

The following medical support system is known. This medical support system detects an abnormality in a subject when having determined that there is a possibility that the subject has developed heart disease or brain disease, on the basis of vital data of the subject, determines the severity of symptoms of the subject when having detected the abnormality in the subject, and specifies a transport destination hospital to which the subject is transported, on the basis of the determined severity of the symptoms of the subject (refer to Patent Literature 1).

### CITATION LIST

### Patent Literature

Patent Literature 1: JP-A-2018-033660

### SUMMARY OF INVENTION

### Problems to be Solved by Invention

Subjects who need to be transported to hospitals may include people who have developed infectious diseases other than heart disease or brain disease. Hence, it is preferable to determine whether or not there is a possibility that a subject has developed an infectious disease and, also when there is a possibility that the subject has developed an infectious disease, detect an abnormality in the subject and specify a transport destination hospital. However, such a method has not been considered at all before.

### Solution to Problems

According to a first aspect of the present invention, a medical support system includes: a vital data acquisition means configured to acquire vital data including blood pressure data, pulse data, electrocardiogram data, and oxygen saturation data of a subject; an abnormality detection means configured to, upon determining that there is a possibility that the subject has developed heart disease, brain disease, or an infectious disease on the basis of the vital data acquired by the vital data acquisition means, detect the possibility that the subject has developed heart disease, brain disease, or an infectious disease, as an abnormality in the subject; a determination means configured to, upon the abnormality detection means detecting the abnormality, determine the severity of symptoms of the subject; a transport destination specification means configured to specify a transport destination hospital to which the subject is transported, on the basis of the severity of the symptoms of the subject determined by the determination means; and a transport destination information output means configured to output transport destination information about the transport destination hospital specified by the transport destination specification means.

According to a second aspect of the present invention, in the medical support system of the first aspect, the determination means determines the severity of the symptoms of the subject on the basis of healthcare information recorded in a storage medium for healthcare of the subject, and the vital data.

According to a third aspect of the present invention, in the medical support system of the second aspect, the determination means converts a determination result into a number for each of a plurality of preset determination items on the basis of the healthcare information and the vital data, calculates a total value of the numbers identified from the determination items, as an index value indicating the severity of the symptoms of the user, and consequently determines the severity of the symptoms of the subject.

According to a fourth aspect of the present invention, the medical support system of the second or third aspect further includes: a question output means configured to output questions for checking the symptoms of the subject in a case where it is impossible to determine the severity of the symptoms of the subject on the basis of the healthcare information and the vital data; and a response data acquisition means configured to accept responses to the questions and acquire response data, in which the determination means determines the severity of the symptoms of the subject on the basis of the response data acquired by the response data acquisition means.

According to a fifth aspect of the present invention, in the medical support system of the fourth aspect, the determination means converts a response result into a number for each of a plurality of preset question items on the basis of the response data, calculates a total value of the numbers identified from the question items, as an index value indicating the severity of the symptoms of the user, and consequently determines the severity of the symptoms of the subject.

According to a sixth aspect of the present invention, the medical support system of any of the first to fifth aspects further includes a person-to-be-transported information transmission means configured to transmit person-to-be-transported information including the healthcare information, the vital data, and data indicating the severity of the symptoms of the subject to the transport destination hospital specified by the transport destination specification means.

According to a seventh aspect of the present invention, the medical support system of any of the first to sixth aspects further includes a notification means configured to, upon the abnormality detection means detecting the abnormality, send notification that the abnormality has been detected in the subject to a preregistered notification destination.

According to an eighth aspect of the present invention, the medical support system of any of the first to seventh aspects further includes a current location identification means configured to identify a current location of the subject, in which in a case where the determination means determines that the severity level of the symptoms of the subject is low, the transport destination specification means selects a hospital nearer to the current location of the subject identified by the current location identification means from a hospital with primary medical care available in a field of a disease matching the symptoms of the subject and a hospital with advanced medical care available in the field of the disease matching the symptoms of the subject, and specifies the hospital as the transport destination hospital.

According to a ninth aspect of the present invention, in the medical support system of the eighth aspect, in a case where the determination means determines that the severity level of the symptoms of the subject is high, the transport destination specification means, upon a difference between time required to reach a hospital with advanced medical care available that is the nearest to the current location of the subject and time required to reach a hospital with primary medical care available that is the nearest to the current location of the subject being longer than a preset threshold, selects a hospital nearer to the current location of the subject identified by the current location identification means from the hospital with primary medical care available and the hospital with advanced medical care available, and specifies the hospital as the transport destination hospital, and upon the difference between the time required to reach the hospital with advanced medical care available that is the nearest to the current location of the subject and the time required to reach the hospital with primary medical care available that is the nearest to the current location of the subject being equal to or shorter than the preset threshold, selects a hospital near to the current location of the subject identified by the current location identification means from the hospital with advanced medical care available, and specifies the hospital as the transport destination hospital.

According to a tenth aspect of the present invention, in the medical support system of any of the first to ninth aspects, the abnormality detection means determines that there is a possibility that the subject has developed heart disease upon a waveform shown in the electrocardiogram data being a preregistered characteristic waveform of heart disease, determines that there is a possibility that the subject has developed brain disease upon a numerical value shown in the blood pressure data and a waveform shown in the pulse data being a preregistered characteristic numerical value of and a preregistered characteristic waveform of brain disease, and determines that there is a possibility that the subject has developed an infectious disease upon a numerical value of the oxygen saturation data being a numerical value suspected of having developed an infectious disease, or in a case where pneumonia is seeable in a chest CT image of the subject.

According to an eleventh aspect of the present invention, in the medical support system of any of the first to tenth aspects, the vital data acquisition means acquires the vital data from a wearable terminal worn by the subject, the wearable terminal having a function of measuring the vital data.

According to a twelfth aspect of the present invention, a medical support system includes: a subject terminal held by a subject; and a monitoring apparatus connected to the subject terminal via a communication line, in which the subject terminal includes: a vital data acquisition means configured to acquire vital data including blood pressure data, pulse data, electrocardiogram data, and oxygen saturation data of the subject; and a vital data transmission means configured to transmit, to the monitoring apparatus, the vital data acquired by the vital data acquisition means, and the monitoring apparatus includes: an abnormality detection means configured to, upon determining that there is a possibility that the subject has developed heart disease, brain disease, or an infectious disease on the basis of the vital data received from the target terminal, detect the possibility that the subject has developed heart disease, brain disease, or an infectious disease, as an abnormality in the subject; a determination means configured to, upon the abnormality detection means detecting the abnormality, determine the severity of symptoms of the subject; a transport destination specification means configured to specify a transport destination hospital to which the subject is transported, on the basis of the severity of the symptoms of the subject determined by the determination means; and a transport destination information transmission means configured to transmit, to the subject terminal, transport destination information about the transport destination hospital specified by the transport destination specification means.

According to a thirteenth aspect of the present invention, in the medical support system of the twelfth aspect, the subject terminal further includes: a transport destination information receiving means configured to receive the transport destination information from the monitoring apparatus; and a transport destination information display means configured to display, on a display device, the transport destination information received by the transport destination information receiving means.

According to a fourteenth aspect of the present invention, in the medical support system of the twelfth or thirteenth aspect, the determination means determines the severity of the symptoms of the subject on the basis of healthcare information recorded in a storage medium for healthcare of the subject, and the vital data.

According to a fifteenth aspect of the present invention, in the medical support system of the fourteenth aspect, the determination means converts a determination result into a number for each of a plurality of preset determination items on the basis of the healthcare information and the vital data, calculates a total value of the numbers identified from the determination items, as an index value indicating the severity of the symptoms of the user, and consequently determines the severity of the symptoms of the subject.

According to a sixteenth aspect of the present invention, in the medical support system of the fourteenth or fifteenth aspect, the subject terminal further includes: a question output means configured to output questions for checking the symptoms of the subject in response to an instruction of the monitoring apparatus; a response data acquisition means configured to accept responses to the questions and acquire response data; and a response data transmission means configured to transmit, to the monitoring apparatus, the response data acquired by the response data acquisition means, and the determination means instructs the subject terminal to output the questions and determines the severity of the symptoms of the subject on the basis of the response data received from the subject terminal in a case where it is impossible to determine the severity of the symptoms of the subject on the basis of the healthcare information and the vital data.

According to a seventeenth aspect of the present invention, in the medical support system of the sixteenth aspect, the determination means converts a response result into a number for each of a plurality of preset question items on the basis of the response data, calculates a total value of the numbers identified from the question items, as an index value indicating the severity of the symptoms of the user, and consequently determines the severity of the symptoms of the subject.

According to an eighteenth aspect of the present invention, in the medical support system of any of the twelfth to seventeenth aspects, the monitoring apparatus further includes a person-to-be-transported information transmission means configured to transmit person-to-be-transported information including the healthcare information, the vital data, and data indicating the severity of the symptoms of the subject to the transport destination hospital specified by the transport destination specification means.

According to a nineteenth aspect of the present invention, in the medical support system of any of the twelfth to eighteenth aspects, the monitoring apparatus further includes a notification means configured to, upon the abnormality detection means detecting the abnormality, send notification that the abnormality has been detected in the subject, to a preregistered notification destination.

According to a twentieth aspect of the present invention, in the medical support system of any of the twelfth to nineteenth aspects, the subject terminal further includes a current location identification means configured to identify a current location of the subject, and in a case where the determination means determines that the severity level of the symptoms of the subject is low, the transport destination specification means selects a hospital nearer to the current location of the subject identified by the current location identification means from a hospital with primary medical care available in a field of a disease matching the symptoms of the subject and a hospital with advanced medical care available in the field of the disease matching the symptoms of the subject, and specifies the hospital as the transport destination hospital.

According to a twenty-first aspect of the present invention, in the medical support system of the twentieth aspect, in a case where the determination means determines that the severity level of the symptoms of the subject is high, the transport destination specification means, upon a difference between time required to reach a hospital with advanced medical care available that is the nearest to the current location of the subject and time required to reach a hospital with primary medical care available that is the nearest to the current location of the subject being longer than a preset threshold, selects a hospital nearer to the current location of the subject identified by the current location identification means from the hospital with primary medical care available and the hospital with advanced medical care available, and specifies the hospital as the transport destination hospital, and upon the difference between the time required to reach the hospital with advanced medical care available that is the nearest to the current location of the subject and the time required to reach the hospital with primary medical care available that is the nearest to the current location of the subject being equal to or shorter than the preset threshold, selects a hospital near to the current location of the subject identified by the current location identification means from the hospital with advanced medical care available, and specifies the hospital as the transport destination hospital.

According to a twenty-second aspect of the present invention, in the medical support system of any of the twelfth to twenty-first aspects, the abnormality detection means determines that there is a possibility that the subject has developed heart disease upon a waveform shown in the electrocardiogram data being a preregistered characteristic waveform of heart disease, determines that there is a possibility that the subject has developed brain disease upon a numerical value shown in the blood pressure data and a waveform shown in the pulse data being a preregistered characteristic numerical value of and a preregistered characteristic waveform of brain disease, and determines that there is a possibility that the subject has developed an infectious disease upon a numerical value of the oxygen saturation data being a numerical value suspected of having developed an infectious disease, or in a case where pneumonia is seeable in a chest CT image of the subject.

According to a twenty-third aspect of the present invention, in the medical support system of any of the twelfth to twenty-second aspects, the vital data acquisition means acquires the vital data from a wearable terminal worn by the subject, the wearable terminal having a function of measuring the vital data.

### Effects of Invention

According to the present invention, not only when there is a possibility that a subject has developed heart disease or brain disease, but also when there is a possibility that the subject has developed an infectious disease, it is possible to determine the severity of symptoms of the subject and specify a transport destination hospital appropriate to the severity of the symptoms. Therefore, when the subject has developed heart disease, brain disease, or an infectious disease, it is possible to quickly determine the severity of the symptoms and specify a transport destination hospital appropriate to the severity of the symptoms.

### BRIEF DESCRIPTION OF DRAWINGS

FIG. 1 is a block diagram illustrating the configuration of an embodiment of a medical support system 10.

FIG. 2 is a block diagram illustrating the configuration of an embodiment of a vital data measuring apparatus 100.

FIG. 3 is a block diagram illustrating the configuration of an embodiment of a user terminal 200.

FIG. 4 is a block diagram illustrating the configuration of an embodiment of a monitoring apparatus 300.

FIG. 5 is a flowchart diagram illustrating the flow of a determination process in a triage process for disease determination.

FIG. 6 is a flowchart diagram illustrating the flow of a determination process in a triage process for infectious disease determination.

FIG. 7 is a flowchart diagram illustrating the flow of a process that is executed by the vital data measuring apparatus 100.

FIG. 8 is a flowchart diagram illustrating the flow of a measurement data transmission process that is executed by the user terminal 200.

FIG. 9 is a flowchart diagram illustrating the flow of a response data transmission process that is executed by the user terminal 200.

FIG. 10 is a flowchart diagram illustrating the flow of a transport destination hospital information output process that is executed by the user terminal 200.

FIG. 11 is a flowchart diagram illustrating the flow of a process that is executed by the monitoring apparatus 300.

### DESCRIPTION OF EMBODIMENTS

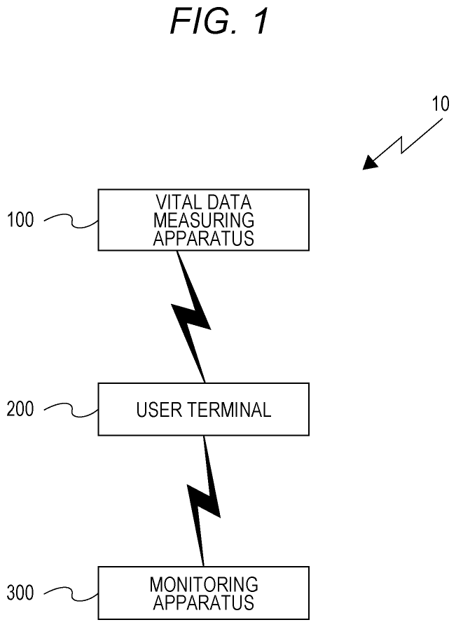

FIG. 1 is a block diagram illustrating the configuration of an embodiment of a medical support system 10 in the embodiment. An object of the medical support system 10 is to increase the survival rate of a user by specifying a hospital appropriate to the severity of symptoms, as a transport destination, when the user has developed heart disease or brain disease such as a cardiac stroke or a cerebral stroke, or an infectious disease. The medical support system 10 includes a vital data measuring apparatus 100 that is worn by a subject targeted for monitoring the health condition and has a function of measuring the subject's vital data, a user terminal 200 held by a user who is the subject, and a monitoring apparatus 300 that monitors data transmitted from the user terminal 200 and detects an abnormality in the user.

The vital data measuring apparatus 100 has functions of acquiring vital data including the user's blood pressure data, pulse data, electrocardiogram data, body temperature data, and oxygen saturation data, and transmitting the acquired vital data to the user terminal 200. For example, a wearable terminal worn by the user on the wrist is used as the vital data measuring apparatus 100. In this wearable terminal, sensors for measuring the blood pressure, pulse, electrocardiogram, body temperature, and oxygen saturation are mounted in a portion that touches the user's body, and it is possible to acquire the user's blood pressure data, pulse data, electrocardiogram data, body temperature data, and oxygen saturation data. A technology for measuring the blood pressure, pulse, electrocardiogram, body temperature, and oxygen saturation of a wearer with a wearable terminal of a type that is wound around the wrist is known; therefore, a description thereof is omitted here.

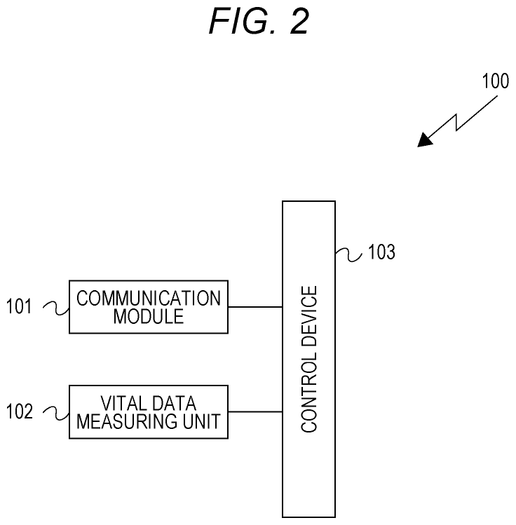

FIG. 2 is a block diagram illustrating the configuration of an embodiment of the vital data measuring apparatus 100 in the embodiment. The vital data measuring apparatus 100 includes a communication module 101, a vital data measuring unit 102, and a control device 103.

The communication module 101 is a module that is connected to the user terminal 200 for the purpose of communication. For example, a module for Bluetooth (registered trademark) communication is used as the communication module 101. In the embodiment, the vital data measuring apparatus 100 is paired in advance by Bluetooth (registered trademark) to the user terminal 200 that is a communication partner, and are in a communicable state.

The vital data measuring unit 102 includes the sensors for measuring the user's blood pressure, pulse, electrocardiogram, body temperature, and oxygen saturation, and peripheral circuits thereof, and outputs the blood pressure data, the pulse data, the electrocardiogram data, the body temperature data, and the oxygen saturation data, which are obtained by the measurements, to the control device 103.

The control device 103 includes a CPU, memory, and other peripheral circuits, and controls the entire vital data measuring apparatus 100. Note that the memory configuring the control device 103 includes, for example, volatile memory such as SDRAM and nonvolatile memory such as flash memory. The volatile memory is used as working memory for developing a program when the CPU executes the program, and buffer memory for temporarily recording data. Moreover, in the nonvolatile memory, data of a program for operating the vital data measuring apparatus 100 and various kinds of data used by the program are recorded.

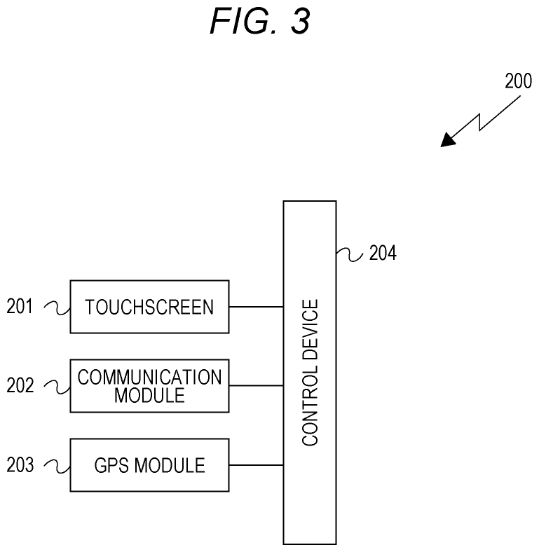

The user terminal 200 is an information terminal held by the subject who is the user in the embodiment. For example, a smartphone, a mobile phone, or a tablet terminal is used as the user terminal 200. FIG. 3 is a block diagram illustrating the configuration of an embodiment in a case where a smartphone is used as the user terminal 200 in the embodiment. In the embodiment, software for the user terminal for executing processes described below is installed on a smartphone, so that the smartphone operates as the user terminal 200 in the embodiment. Note that a program of the software for the user terminal is assumed to be provided by being published on a server connected via the Internet, downloaded and obtained by the user himself/herself or a person concerned such as the user's family doctor or family member, and preinstalled on the user terminal 200. Alternatively, the program of the software for the user terminal may be provided by being recorded in a recording medium such as a CD-ROM or a DVD-ROM.

The user terminal 200 includes a touchscreen 201, a communication module 202, a GPS module 203, and a control device 204.

The touchscreen 201 is an electronic component in which a display device such as a liquid crystal panel and a position input device such as a touch pad are combined, and is an input device that can operate equipment by pressing a display on the screen. For example, an operator of the user terminal 200 can operate the user terminal 200 by touching or sliding a display element such as a button or menu displayed on the liquid crystal panel by use of the finger or a touch pen. The touchscreen 201 detects an operation such as a touch or slide by the operator, and outputs a detection signal for the touch or slide to the control device 204.

The communication module 202 includes a module for connecting the user terminal 200 to the Internet via a LAN or a mobile phone communication network in a wireless or wired manner, and a module for near-field communication for communicating with the vital data measuring apparatus 100. The user terminal 200 can communicate with the monitoring apparatus 300 by connecting to the Internet via the communication module 202. Moreover, the user terminal 200 can communicate with the vital data measuring apparatus 100, which has been paired in advance to the user terminal 200, via the communication module 202.

The GPS module 203 is a module for communicating with GPS satellites and receiving positioning information for identifying the current location from the GPS satellites, and includes an antenna and a control circuit for communicating with the GPS satellites.

The control device 204 includes a CPU, memory, and other peripheral circuits, and controls the entire user terminal 200. Note that the memory configuring the control device 204 includes, for example, volatile memory such as SDRAM and nonvolatile memory such as flash memory. The volatile memory is used as working memory for developing a program when the CPU executes the program, and buffer memory for temporarily recording data. Moreover, program data of firmware for operating the user terminal 200 and software for operating various applications are recorded in the nonvolatile memory. In the embodiment, the program of the above-mentioned software for the user terminal is recorded in the nonvolatile memory.

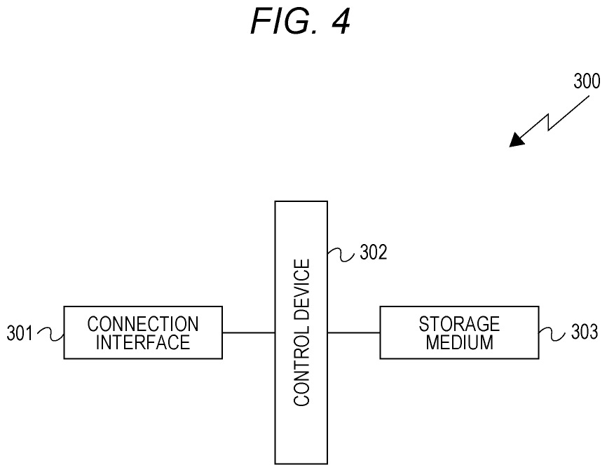

The monitoring apparatus 300 is an apparatus connected to the Internet. For example, a personal computer or a server is used as the monitoring apparatus 300. FIG. 4 is a block diagram illustrating the configuration of an embodiment in a case where a server apparatus is used as the monitoring apparatus 300 in the embodiment. The monitoring apparatus 300 includes a connection interface 301, a control device 302, and a storage medium 303.

The connection interface 301 is an interface for connecting the monitoring apparatus 300 to a communication line for, for example, the Internet. For example, a wired LAN module for wired connection to the Internet, or a wireless LAN module for wireless connection to the Internet is used as the connection interface 301. In the embodiment, the monitoring apparatus 300 communicates with the user terminal 200 via the connection interface 301.

The control device 302 includes a CPU, memory, and other peripheral circuits, and controls the entire monitoring apparatus 300. Note that the memory configuring the control device 302 is, for example, volatile memory such as SDRAM. This memory is used as working memory for developing a program when the CPU executes the program, and buffer memory for temporarily recording data. For example, data read via the connection interface 301 is temporarily recorded in the buffer memory.

The storage medium 303 is a storage medium for recording, for example, various kinds of data to be stored in the monitoring apparatus 300, and data of a program to be executed by the control device 302. For example, an HDD (Hard Disk Drive) or an SSD (Solid State Drive) is used as the storage medium 303. Note that the data of the program to be recorded in the storage medium 303 is provided by being recorded in a recording medium such as a CD-ROM or a DVD-ROM, or is provided via a network, and the control device 302 can execute the program by installing the data of the program acquired by the operator onto the storage medium 303.

In the medical support system 10 in the embodiment, the user who holds the user terminal 200 registers information about his/her healthcare to use the medical support system 10. For this purpose, the user starts the software for the user terminal on the user terminal 200, and registers the information about his/her healthcare on an information registration screen displayed on the touchscreen 201. Examples of the healthcare information about the user's healthcare include data indicating name, sex, date of birth, height, weight, blood type, smoking or non-smoking, past medical history, current medications, and the user's health condition. The data indicating the user's health condition includes data related to the occurrence of brain disease, heart disease, or an infectious disease. Examples of the data related to the occurrence of brain disease or heart disease include the value of LDL cholesterol and the value of triglyceride, which are obtained by a pre-examination. Moreover, the data related to the occurrence of an infectious disease includes information indicating the presence or absence of malaise, information indicating the presence or absence of breathlessness, information indicating the presence or absence of diarrhea and/or vomiting, information indicating whether or not pneumonia can be seen in a chest CT image, information about vaccination, and information about close contact with an infected person.

When the user inputs the healthcare information on the information registration screen and instructs the registration of the healthcare information, the control device 204 transmits the inputted healthcare information to the monitoring apparatus 300. In the monitoring apparatus 300, when having received the healthcare information from the user terminal 200, the control device 302 issues information for uniquely identifying the user, for example, a user ID, and records the user ID and the healthcare information in association with each other in the storage medium 303. Moreover, the control device 302 transmits the issued user ID to the user terminal 200. In the user terminal 200, the control device 204 records, in the memory, the user ID received from the monitoring apparatus 300. As a result, the registration of the user information in the monitoring apparatus 300 is completed, and the user can use the medical support system 10.

Note that if the user's own healthcare information is changed, the user can display the information registration screen to correct the healthcare information as appropriate. The corrected healthcare information is transmitted to the monitoring apparatus 300, and the control device 302 updates the user's healthcare information to the corrected information.

In order to use the medical support system 10, the user wears the vital data measuring apparatus 100 that has been paired in advance to the user terminal 200. In the embodiment, a wearable terminal of a type that is worn around the wrist is assumed; therefore, the user wears the vital data measuring apparatus 100 on the wrist. As a result, the vital data measuring apparatus 100 starts measuring the user's vital data.

The control device 103 of the vital data measuring apparatus 100 acquires the user's vital data inputted through the vital data measuring unit 102, and transmits the acquired vital data to the user terminal 200 through the communication module 101. Note that the transmission timing of the vital data is not particularly limited, but the control device 103 may be configured in such a manner as to always continue the acquisition and transmission of the vital data, or to transmit the vital data to the user terminal 200 at preset predetermined time intervals, for example, every minute, every 5 minutes, and every 10 minutes. Moreover, it may be configured in such a manner that an operator such as the user, or the user's family member or family doctor can freely set the time intervals for transmitting the vital data.

In the user terminal 200, when having received the vital data from the vital data measuring apparatus 100, the control device 204 transmits measurement data including the received vital data, the information for identifying the user, for example, the above-mentioned user ID, and current location information indicating the current location of the user terminal 200, to the monitoring apparatus 300 through the communication module 202. Note that the control device 204 acquires the current location information indicating the current location of the user terminal 200 on the basis of the output from the GPS module 203.

In the monitoring apparatus 300, when having received the measurement data from the user terminal 200, the control device 302 determines whether or not there is a possibility that the user has developed heart disease, brain disease, or an infectious disease on the basis of the vital data included in the received measurement data and, if determining that there is a possibility that the user has developed heart disease, brain disease, or an infectious disease, detects an abnormality in the user. Here, a process of detecting an abnormality in the user is described.

In order to detect an abnormality in the user, data of the characteristic electrocardiogram waveform of a patient who has developed heart disease, the numerical value of characteristic blood pressure of a patient who has developed brain disease, and the characteristic pulse waveform of a patient who has developed brain disease are prerecorded in the storage medium 303. When having detected that the waveform shown in the electrocardiogram data included in the vital data is the characteristic waveform of heart disease preregistered in the storage medium 303, the control device 302 determines that there is a possibility that the user has developed heart disease, and detects an abnormality in the user. Moreover, when the numeric value of the blood pressure data and the waveform shown in the pulse data, which are included in the vital data, are the characteristic numeric value and waveform of brain disease preregistered in the storage medium 303, the control device 302 determines that there is a possibility that the user has developed brain disease, and detects an abnormality in the user. Moreover, when the numerical value of the oxygen saturation data included in the vital data is a numerical value that is preregistered in the storage medium 303 and is suspected of having developed an infectious disease, or if pneumonia can be seen in a chest CT image of the user, the control device 302 determines that there is a possibility that the user has developed an infectious disease, and detects an abnormality in the user. Note that it is assumed that a radiologist or an image diagnosis system makes the determination on the presence or absence of pneumonia based on a chest CT image of the user, on the basis of a chest CT image of the user captured in advance, and a determination result thereof is recorded as “information indicating whether or not pneumonia can be seen in the chest CT image” included in the above-mentioned data related to the occurrence of an infectious disease.

If determining that there is a possibility that the user has developed heart disease or brain disease and consequently detecting an abnormality in the user, the control device 302 executes a triage process for disease determination described below. The control device 302 then determines which hospital the user should be transported to, a hospital with advanced medical care available that provides advanced medical care in a field of the disease that the user has developed, or a hospital with primary medical care available that provides primary medical care in the field of the disease that the user has developed, and decides a transport destination hospital for the user. Note that, in the embodiment, it is configured in such a manner that even if the user can be treated at the hospital with primary medical care available, when the hospital with advanced medical care available is nearer to the user's current location than the hospital with primary medical care available, the user is transported to the hospital with advanced medical care available to increase the survival rate.

Moreover, if determining that there is a possibility that the user has developed an infectious disease and consequently detecting an abnormality in the user, the control device 302 executes a triage process for infectious disease determination described below. If there is a possibility that the user has developed an infectious disease, then the control device 302 determines which hospital the user should be transported to, a hospital with advanced medical care available that provides advanced medical care for infectious diseases, a hospital with secondary medical care available that provides secondary medical care for infectious diseases, or a hospital with primary medical care available that provides primary medical care for infectious diseases, and decides a transport destination hospital for the user. Note that the hospital with advanced medical care available that provides advanced medical care for infectious diseases is assumed to be, for example, an infection control hospital and be a hospital with a heart-lung machine, the hospital with secondary medical care available that provides secondary medical care for infectious diseases is assumed to be, for example, an infection control hospital, and the hospital with primary medical care available that provides primary medical care for infectious diseases is assumed to be a hospital other than the hospitals with advanced and secondary medical care available.

In order to decide the transport destination hospital by the triage process for disease determination or the triage process for infectious disease determination, data of hospital information about hospitals is prerecorded in the storage medium 303. The hospital information includes information such as hospital name, location information (longitude and latitude) representing the address of a hospital, availability of advanced/primary medical care for each disease field, availability of advanced/secondary/primary medical care for infectious diseases, emergency medical response hours, email address, telephone number, information about a specialist of each disease field, the number of beds and availability thereof, availability of infectious disease beds, availability of respiratory devices and heart-lung machines, and the number of operating rooms and availability thereof. Note that the information about a specialist of each disease field includes the name, specialized field, and mobile phone number of the doctor. Moreover, the number of beds includes the number of beds and availability thereof in each specialized care unit such as a general care unit, a stroke intensive care unit, a respiratory intensive care unit, and a pediatric intensive care unit.

The hospital information is inputted on an external terminal such as a terminal that is operated by an administrator of the medical support system 10 or a terminal that is installed in each hospital, transmitted to the monitoring apparatus 300, and recorded in the storage medium 303 by the control device 302. Moreover, the hospital information is updated to the latest information by use of the external terminal whenever necessary.

Firstly, a description is given of the triage process for disease determination that is executed when it has been determined that there is a possibility that the user has developed heart disease or brain disease. In the embodiment, the triage process for disease determination includes an automatic triage process in which the control device 302 determines the severity of symptoms of the user on the basis of the user's healthcare information and vital data and decides a transport destination hospital, and a question-type triage process in which questions for checking the user's family member or an emergency medical technician for the symptoms of the user are outputted onto the user terminal 200, and the control device 302 determines the severity of the symptoms of the user on the basis of response data obtained on the basis of the questions and decides a transport destination hospital.

In the embodiment, if the control device 302 detects an abnormality in the user, and if the control device 302 can execute the automatic triage process based on the user's healthcare information and vital data, the control device 302 executes the automatic triage process and quickly decides a transport destination hospital for the user. On the other hand, if the healthcare information and the vital data information are insufficient and a transport destination hospital for the user cannot be decided by the automatic triage process, the control device 302 outputs the questions for checking the symptoms of the user through the user terminal 200, waits until receiving response data from the user terminal 200, executes the question-type triage process, and decides a transport destination hospital appropriate to the symptoms of the user.

The control device 302 reads the user's healthcare information from the storage medium 303 on the basis of the user ID included in the measurement data received from user terminal 200. The control device 302 then executes the automatic triage process on the basis of the healthcare information that has been read out and the vital data received from the user terminal 200, and determines which hospital the user should be transported to, a hospital with advanced medical care available or a hospital with primary medical care available.

In the automatic triage process in the triage process for disease determination, the control device 302 converts determination results of the following determination items (A1) to (A10) into numbers, and calculates a total value of the numbers as an index value indicating the severity of the symptoms of the user. In the embodiment, an example is described in which scores corresponding to the determination results are assigned to the following determination items (A1) to (A10), and a total value of the scores for the determination items (A1) to (A10) is calculated as an index value indicating the severity of the symptoms of the user in the automatic triage process. Note that the scores are set in such a manner that the total value increases with increasing possibility of becoming severe when the user develops brain disease or heart disease; therefore, a higher total value indicates a higher possibility that the severity level of the symptoms of the user is high.
(A1) If the value of LDL cholesterol is 140 mg/dL or more on the basis of the data indicating the user's health condition, the score is +0.5.
(A2) If the value of triglyceride is 150 mg/dL or more on the basis of the data indicating the user's health condition, the score is +0.5.
(A3) If the user is a smoker, the score is +2.5.
(A4) If the user's past medical history includes cerebral infarction, the score is +0.5.
(A5) If the age of the user is 70 or older, the score is +0.5.
(A6) If the value of HB1AC is 6.5 mg/dL or more and the user's past medical history includes diabetes on the basis of the data indicating the user's health condition, the score is +0.5.
(A7) If the value of the BMI calculated on the basis of the user's height and weight is 25 or more, the score is +0.5.
(A8) If the user's past medical history includes heavy drinking, the score is +0.25.
(A9) If the blood pressure at the time of detection of an abnormality in the user is 140/90 mmhg or more, the score is +1.
(A10) If the waveform of arrhythmia like atrial fibrillation is occurring in electrocardiogram data produced when the abnormality in the user is detected, the score is +1.

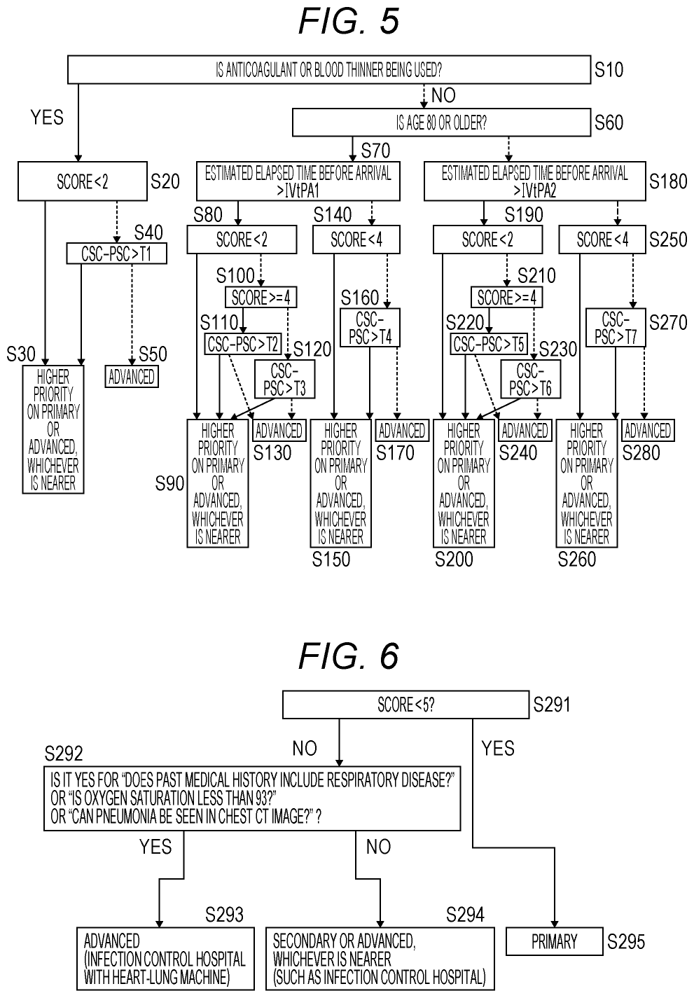

The control device 302 executes a process in a flow illustrated in FIG. 5, and consequently, specifies a transport destination appropriate to the severity of the symptoms of the user on the basis of the total value of the scores based on the determination items (A1) to (A10) calculated in the above process. The process illustrated in FIG. 5 is described below.

In the question-type triage process in the triage process for disease determination, the control device 302 obtains response data to the following question items (B1) to (B12), and specifies a transport destination hospital for the user on the basis of the response data. Note that the control device 302 instructs the user terminal 200 to output questions for checking the symptoms of the user to execute the question-type triage process if a determination cannot be made on any of the above determination items (A1) to (A10) in the automatic triage process.

When the monitoring apparatus 300 instructs the user terminal 200 to output the questions, the control device 204 outputs the question contents corresponding to the following question items (B1) to (B12) to the touchscreen 201 and displays the question contents thereon. After accepting the input of a response to each of the question items, the control device 204 transmits response data indicating the response content to the monitoring apparatus 300. Note that the question contents corresponding to the following question items (B1) to (B12) may be prerecorded in the memory included in the user terminal 200, or may be acquired from the monitoring apparatus 300. Note that, in the following questions, the patient means the user who is the subject. Moreover, the following question contents are not limited to the following words as long as they are contents to which substantially the same responses can be obtained. Moreover, in terms of items that can specify responses on the basis of the user's healthcare information among the following question items, the response data may be acquired on the basis of the user's healthcare information without asking the questions.
(B1) Is the patient using an anticoagulant or blood thinner?
(B2) What is the age of the patient?
(B3) Did anyone see the patient at the time of occurrence?
(B4) What is the time when the patient was fine for the last time?
(B5) Does the patient have any symptoms of facial paralysis?
(B6) Does the patient have any symptoms of paralysis in the arms?
(B7) Does what the patient says make sense?
(B8) Ask the patient the names of three general objects. Was he/she able to name them?
(B9) Ask the patient: “Please show me two of your fingers.” Was the patient able to do it properly?
(B10) Does the patient have any symptoms of eye deviation?
(B11) Ask the patient: “Do you have any subjective symptoms?” Does the patient have any subjective symptoms?
(B12) Ask the patient: “Whose arm is this?” Was the patient able to answer correctly?

An operator such as a family member or an emergency medical technician selects either “yes” or “no” to respond to the question item (B1). The operator selects either “80 or older” or “younger than 80” to respond to the question item (B2). If there is a person who saw the user at the time of occurrence, the time when the person saw the occurrence is given as a response to the question item (B3). The time that can be confirmed as the last time the patient was fine is given as a response to the question item (B4). The operator selects either “without symptoms” or “with mild symptoms” to respond to the question item (B5). The operator selects any of “without symptoms,” “with symptoms,” or “with severe symptoms” to respond to the question item (B6). The operator selects either “yes” or “no” to respond to the question item (B7). The operator selects either “yes” or “no” to respond to the question item (B8). The question item (B9) is outputted if “without symptoms” is selected for (B6), and the operator selects either “able to do properly” or “unable to do properly” to respond to the question item (B9). The operator selects any of “without symptoms,” “with symptoms in one eye,” or “with symptoms in two eyes” to respond to the question item (B10). The question item (B11) is outputted if “with symptoms” or “with severe symptoms” is selected for (B6), and the operator selects either “without symptoms” or “with symptoms” to respond to the question item (B11). The question item (B12) is outputted if “with symptoms” or “with severe symptoms” is selected for (B6), and the operator selects either “yes” or “no” to respond to the question item (B12).

When having received response data to the above question items (B1) to (B12), the control device 302 converts response results of the response data to the question items (B5) to (B12) into numbers, and calculates a total value of the numbers as an index value indicating the severity of the symptoms of the user. In the embodiment, as described below, an example is described in which scores corresponding to response contents are assigned to the question items (B5) to (B10), and a total value of the scores for the question items (B5) to (B10) is calculated as an index value indicating the severity of the symptoms of the user in the question-type triage process. Note that the scores are set in such a manner that the total value increases with increasing possibility of becoming severe when the user develops brain disease or heart disease; therefore, a higher total value indicates a higher possibility that the severity level of the symptoms of the user is high.

If the response to the question item (B5) is “with mild symptoms,” the score is +1.

If the response to the question item (B6) is “with symptoms,” the score is +1, and if the response is “with severe symptoms,” the score is +2.

If the response to the question item (B7) is “no,” the score is +1.

If the response to the question item (B8) is “no,” the score is +1.

If the response to the question item (B9) is “unable to do properly,” the score is +1.

If the response to the question item (B10) is “with symptoms in one eye,” the score is +1, and if the response is “with symptoms in two eyes,” the score is +2.

If the response to the question item (B11) is “with symptoms,” the score is +1.

If the response to the question item (B12) is “no,” the score is +1.

The control device 302 executes the process in the flow illustrated in FIG. 5 and, therefore, specifies a transport destination appropriate to the severity of the symptoms of the user on the basis of the response contents to the questions (B1) to (B4) and the total value of the scores based on the question items (B5) to (B12), which were calculated in the above process.

Next, a determination process that is performed to specify a transport destination hospital in the automatic triage process and the question-type triage process is described using the flow illustrated in FIG. 5. Note that in FIG. 5, arrows in solid lines indicate affirmative judgments (YES), and arrows in dotted lines indicate negative judgments (NO).

In step S10, the control device 302 judges whether or not the user is taking an anticoagulant or blood thinner. In the case of the automatic triage, the control device 302 judges whether or not the user is taking an anticoagulant or blood thinner on the basis of the information on the current medications included in the user's healthcare information. Moreover, in the case of the question-type triage, the control device 302 judges whether or not the user is taking an anticoagulant or blood thinner on the basis of response data to the above question item (B1). If an affirmative judgment is made in step S10, the procedure moves on to step S20.

In step S20, the control device 302 judges whether or not the total value of the scores in the above-mentioned automatic triage process or question-type triage process is less than two. Note that, in step S20, it is assumed that if the total value of the scores of the user who is taking an anticoagulant or blood thinner in the automatic triage process or question-type triage process is less than two, the severity level of the symptoms of the user can be determined to be low, and if the total value of the scores is two or more, the severity level of the symptoms of the user can be determined to be high.

If an affirmative judgment is made in step S20, the procedure moves on to step S30, and the control device 302 determines that the user should be transported to a hospital with primary medical care available, or a hospital with advanced medical care available, in a field of a disease that the user has developed, whichever is nearer. As a result, it can be determined that the user having a high possibility that the symptoms are not severe (the severity level is low) should be transported to the hospital with primary medical care available, or the hospital with advanced medical care available, whichever is nearer, with a higher priority on transporting the user to a hospital as soon as possible.

On the other hand, if a negative judgment is made in step S20, the procedure moves on to step S40. In step S40, the control device 302 judges whether or not a difference between time required to reach a hospital with advanced medical care available that is the nearest to the user's current location (CSC) and time required to reach a hospital with primary medical care available that is the nearest to the user's current location (PSC) is longer than a preset threshold T1. The time required to reach the hospital with advanced medical care available that is the nearest to the user's current location (CSC) and the time required to reach the hospital with primary medical care available that is the nearest to the user's current location (PSC) are determined on the basis of the information on the user's current location and the location information representing the addresses of hospitals included in the hospital information.

In view of a situation in which the user is taking an anticoagulant or a blood thinner and the total value of the scores is two or more, there is a possibility that the severity level of the symptoms of the user is high, and therefore, it is considered that it is preferable to transport the user to the hospital with advanced medical care available. However, even in such a situation, if the difference between the time required to reach the hospital with advanced medical care available that is the nearest to the user's current location (CSC) and the time required to reach the hospital with primary medical care available that is the nearest to the user's current location (PSC) is a predetermined time or longer, there is a case in which a higher priority should be placed on quick transport to a hospital without being bound by transport to a hospital with advanced medical care available. The value of the threshold T1 is set in view of such circumstances, and it is assumed in the embodiment that, for example, 30 minutes is set as the threshold T1. Note that the threshold T1 is set at an optimum value in view of the above circumstances, and is not limited to 30 minutes.

If an affirmative judgment is made in step S40, the procedure moves on to step S30, and the control device 302 determines that the user should be transported to the hospital with primary medical care available, or the hospital with advanced medical care available, in the field of the disease that the user has developed, whichever is nearer. As a result, if the total value of the scores is high and there is a high possibility that the symptoms are severe (the severity level is high), but the difference between the time required to reach the nearest hospital with advanced medical care available (CSC) and the time required to reach the nearest hospital with primary medical care available (PSC) is longer than the predetermined threshold T1, it is possible to determine that the user should be transported to the hospital with primary medical care available, or the hospital with advanced medical care available, whichever is nearer, to place a higher priority on quickly transporting the user to a hospital.

In contrast, if a negative judgment is made in step S40, the procedure moves on to step S50, and the control device 302 determines that the user should be transported to the hospital with advanced medical care available in the field of the disease that the user has developed. As a result, if the difference between the time required to reach the nearest hospital with advanced medical care available (CSC) and the time required to reach the nearest hospital with primary medical care available (PSC) is short, it is possible to determine that the user having a high total value of the scores and a high possibility that the symptoms are severe should be transported to the hospital with advanced medical care available that can provide treatment appropriate to the severity of the symptoms of the user.

If a negative judgment is made in step S10, the procedure moves on to step S60. In step S60, the control device 302 judges whether or not the age of the user is 80 or older. In the case of the automatic triage, the control device 302 judges whether or not the age of the user is 80 or older on the basis of the information on the date of birth included in the user's healthcare information. Moreover, in the case of the question-type triage, the control device 302 judges whether or not the age of the user is 80 or older on the basis of response data to the above question item (B2). If an affirmative judgment is made in step S60, the procedure moves on to step S70.

In step S70, the control device 302 calculates estimated elapsed time from the detection of an abnormality in the user to arrival at the hospital, and determines whether or not the calculated estimated elapsed time is longer than a threshold IVtPA1. The estimated elapsed time from the detection of an abnormality in the user to arrival at the hospital is calculated by the following equation (1). Note that the threshold IVtPA1 is set on the basis of allowable time before administration of a thrombolytic agent (t-PA). The allowable time is generally within 270 minutes. However, since there is a clinical trial result the allowable time for age 80 or older is less than 180 minutes, the threshold IVtPA1 is set at 180 minutes here.

Estimated elapsed time before arrival=current time−time of detection of an abnormality in the user+shortest arrival time required to reach the hospital with primary or advanced medical care available in a field of a disease that the user has developed (1)

Note that, in equation (1), the time of detection of an abnormality in the user is the time when an abnormality in the user is detected on the basis of the user's vital data in the case of the automatic triage. Moreover, in the case of the question-type triage, the time of detection of an abnormality in the user is the time acquired as response data to the above question item (B3) and, if there is no response to the above question item (B3) and response data to the question item (B4) has been obtained, is the time acquired as the response data to the question item (B4).

If an affirmative judgment is made in step S70, the procedure moves on to step S80, and the control device 302 judges whether or not the total value of the scores in the above-mentioned automatic triage process or question-type triage process is less than two. Note that it is assumed that if, in step S80, in terms of a user aged 80 or older who is not taking an anticoagulant or a blood thinner, the estimated elapsed time before arrival is longer than the allowable time before administration of a thrombolytic agent (t-PA), it can be determined that the severity level of the symptoms of the user is low in a case where the total value of the scores in the automatic triage process or in the question-type triage process is less than two, and it can be determined that the severity level of the symptoms of the user is high in a case where the total value of the scores is two or more.

If an affirmative judgment is made in step S80, the procedure moves on to step S90, and the control device 302 determines that the user should be transported to the hospital with primary medical care available, or the hospital with advanced medical care available, in the field of the disease that the user has developed, whichever is nearer. As a result, it is possible to determine that the user having a high possibility that the symptoms are not severe should be transported to the hospital with primary medical care available, or the hospital with advanced medical care available, whichever is nearer, with a higher priority on transporting the user to a hospital as soon as possible.

On the other hand, if a negative judgment is made in step S80, the procedure moves on to step S100. In step S100, the control device 302 judges whether or not the total value of the scores in the above-mentioned automatic triage process or question-type triage process is four or more. If an affirmative judgment is made in step S100, the procedure moves on to step S110, and the control device 302 judges whether or not the difference between the time required to reach a hospital with advanced medical care available that is the nearest to the user's current location (CSC) and the time required to reach a hospital with primary medical care available that is the nearest to the user's current location (PSC) is longer than a preset threshold T2.

In view of a situation in which the user is not taking an anticoagulant or a blood thinner, the age is 80 or older, the estimated elapsed time before arrival is longer than 180 minutes, and the total value of the scores is four or more, there is a possibility that the severity level of the symptoms of the user is high, and therefore, it is considered that it is preferable to transport the user to the hospital with advanced medical care available. However, even in such a situation, if the difference between the time required to reach the hospital with advanced medical care available that is the nearest to the user's current location (CSC) and the time required to reach the hospital with primary medical care available that is the nearest to the user's current location (PSC) is a predetermined time or longer, there is a case in which a higher priority should be placed on quick transport to a hospital without being bound by transport to a hospital with advanced medical care available. The value of the threshold T2 is set in view of such circumstances, and it is assumed in the embodiment that, for example, 60 minutes is set as the threshold T2. Note that the threshold T2 is set at an optimum value in view of the above circumstances, and is not limited to 60 minutes.

If an affirmative judgment is made in step S110, the procedure moves on to step S90, and the control device 302 determines that the user should be transported to the hospital with primary medical care available, or the hospital with advanced medical care available, in the field of the disease that the user has developed, whichever is nearer. As a result, if the total value of the scores is high and there is a high possibility that the symptoms are severe, but the difference between the time required to reach the nearest hospital with advanced medical care available (CSC) and the time required to reach the nearest hospital with primary medical care available (PSC) is longer than the threshold T2, it is possible to determine that the user should be transported to the hospital with primary medical care available, or the hospital with advanced medical care available, whichever is nearer, to place a higher priority on quickly transporting the user to a hospital.

In contrast, if a negative judgment is made in step S110, the procedure moves on to step S130, and the control device 302 determines that the user should be transported to the hospital with advanced medical care available in the field of the disease that the user has developed. As a result, if the difference between the time required to reach the nearest hospital with advanced medical care available (CSC) and the time required to reach the nearest hospital with primary medical care available (PSC) is short, it is possible to determine that the user having a high total value of the scores and a high possibility that the symptoms are severe should be transported to the hospital with advanced medical care available that can provide treatment appropriate to the severity of the symptoms of the user.

On the other hand, if a negative judgment is made in step S100, the procedure moves on to step S120, and the control device 302 judges whether or not the difference between the time required to reach a hospital with advanced medical care available that is the nearest to the user's current location (CSC) and the time required to reach a hospital with primary medical care available that is the nearest to the user's current location (PSC) is longer than a preset threshold T3.

In view of a situation in which the user is not taking an anticoagulant or a blood thinner, the age is 80 or older, the estimated elapsed time before arrival is longer than 180 minutes, and the total value of the scores is equal to or greater than two and less than four, there is a possibility that the severity level of the symptoms of the user is high, and therefore, it is considered that it is preferable to transport the user to the hospital with advanced medical care available. However, even in such a situation, if the difference between the time required to reach the hospital with advanced medical care available that is the nearest to the user's current location (CSC) and the time required to reach the hospital with primary medical care available that is the nearest to the user's current location (PSC) is a predetermined time or longer, there is a case in which a higher priority should be placed on quick transport to a hospital without being bound by transport to a hospital with advanced medical care available. The value of the threshold T3 is set in view of such circumstances, and it is assumed in the embodiment that, for example, 30 minutes is set as the threshold T3. Note that the threshold T3 is set at an optimum value in view of the above circumstances, and is not limited to 30 minutes.

If an affirmative judgment is made in step S120, the procedure moves on to step S90, and the control device 302 determines that the user should be transported to the hospital with primary medical care available, or the hospital with advanced medical care available, in the field of the disease that the user has developed, whichever is nearer. As a result, if the total value of the scores is high and there is a high possibility that the symptoms are severe, but the difference between the time required to reach the nearest hospital with advanced medical care available (CSC) and the time required to reach the nearest hospital with primary medical care available (PSC) is longer than the threshold T3, it is possible to determine that the user should be transported to the hospital with primary medical care available, or the hospital with advanced medical care available, whichever is nearer, to place a higher priority on quickly transporting the user to a hospital.

In contrast, if a negative judgment is made in step S120, the procedure moves on to step S130, and the control device 302 determines that the user should be transported to the hospital with advanced medical care available in the field of the disease that the user has developed. As a result, if the difference between the time required to reach the nearest hospital with advanced medical care available (CSC) and the time required to reach the nearest hospital with primary medical care available (PSC) is short, it is possible to determine that the user having a high total value of the scores and a high possibility that the symptoms are severe should be transported to the hospital with advanced medical care available that can provide treatment appropriate to the severity of the symptoms of the user.

If a negative judgment is made in step S70, the procedure moves on to step S140, and the control device 302 judges whether or not the total value of the scores in the above-mentioned automatic triage process or question-type triage process is less than four. Note that it is assumed that if, in step S140, in terms of a user aged 80 or older who is not taking an anticoagulant or a blood thinner, the estimated elapsed time before arrival is equal to or shorter than the allowable time before administration of a thrombolytic agent (t-PA), it can be determined that the severity level of the symptoms of the user is low in a case where the total value of the scores in the automatic triage process or in the question-type triage process is less than four, and it can be determined that the severity level of the symptoms of the user is high in a case where the total value of the scores is four or more.

If an affirmative judgment is made in step S140, the procedure moves on to step S150, and the control device 302 determines that the user should be transported to the hospital with primary medical care available, or the hospital with advanced medical care available, in the field of the disease that the user has developed, whichever is nearer. As a result, it is possible to determine that the user having a high possibility that the symptoms are not severe should be transported to the hospital with primary medical care available, or the hospital with advanced medical care available, whichever is nearer, with a higher priority on transporting the user to a hospital as soon as possible.

On the other hand, if a negative judgment is made in step S140, the procedure moves on to step S160. In step S160, the control device 302 judges whether or not the difference between the time required to reach the hospital with advanced medical care available that is the nearest to the user's current location (CSC) and the time required to reach the hospital with primary medical care available that is the nearest to the user's current location (PSC) is longer than a preset threshold T4.

In view of a situation in which the user is not taking an anticoagulant or a blood thinner, the age is 80 or older, the estimated elapsed time before arrival is 180 minutes or less, and the total value of the scores is four or more, there is a possibility that the severity level of the symptoms of the user is high, and therefore, it is considered that it is preferable to transport the user to the hospital with advanced medical care available. However, even in such a situation, if the difference between the time required to reach the hospital with advanced medical care available that is the nearest to the user's current location (CSC) and the time required to reach the hospital with primary medical care available that is the nearest to the user's current location (PSC) is a predetermined time or longer, there is a case in which a higher priority should be placed on quick transport to a hospital without being bound by transport to a hospital with advanced medical care available. The value of the threshold T4 is set in view of such circumstances, and it is assumed in the embodiment that, for example, 30 minutes is set as the threshold T4. Note that the threshold T4 is set at an optimum value in view of the above circumstances, and is not limited to 30 minutes.

If an affirmative judgment is made in step S160, the procedure moves on to step S150, and the control device 302 determines that the user should be transported to the hospital with primary medical care available, or the hospital with advanced medical care available, in the field of the disease that the user has developed, whichever is nearer. As a result, if the total value of the scores is high and there is a high possibility that the symptoms are severe, but the difference between the time required to reach the nearest hospital with advanced medical care available (CSC) and the time required to reach the nearest hospital with primary medical care available (PSC) is longer than the threshold T4, it is possible to determine that the user should be transported to the hospital with primary medical care available, or the hospital with advanced medical care available, whichever is nearer, to place a higher priority on quickly transporting the user to a hospital.

In contrast, if a negative judgment is made in step S160, the procedure moves on to step S170, and the control device 302 determines that the user should be transported to the hospital with advanced medical care available in the field of the disease that the user has developed. As a result, if the difference between the time required to reach the nearest hospital with advanced medical care available (CSC) and the time required to reach the nearest hospital with primary medical care available (PSC) is short, it is possible to determine that the user having a high total value of the scores and a high possibility that the symptoms are severe should be transported to the hospital with advanced medical care available that can provide treatment appropriate to the severity of the symptoms of the user.

Next, a process in the case where a negative judgment is made in step S60 is described. If a negative judgment is made in step S60, the procedure moves on to step S180. In step S180, the control device 302 calculates the estimated elapsed time from the detection of an abnormality in the user to arrival at the hospital by using equation (1), and determines whether or not the calculated estimated elapsed time is longer than a threshold IVtPA2. As described above, since the general allowable time before administration of a thrombolytic agent (t-PA) is within 270 minutes, the threshold IVtPA2 in step S180 is set at 270 minutes.

If an affirmative judgment is made in step S180, the procedure moves on to step S190, and the control device 302 judges whether or not the total value of the scores in the above-mentioned automatic triage process or question-type triage process is less than two. Note that it is assumed that if, in step S190, in terms of a user aged younger than 80 who is not taking an anticoagulant or a blood thinner, the estimated elapsed time before arrival is longer than the allowable time before administration of a thrombolytic agent (t-PA), it is possible to determine that the severity level of the symptoms of the user is low in a case where the total value of the scores in the automatic triage process or in the question-type triage process is less than two, and it is possible to determine that the severity level of the symptoms of the user is high in a case where the total value of the scores is two or more.

If an affirmative judgment is made in step S190, the procedure moves on to step S200, and the control device 302 determines that the user should be transported to the hospital with primary medical care available, or the hospital with advanced medical care available, in the field of the disease that the user has developed, whichever is nearer. As a result, it is possible to determine that the user having a high possibility that the symptoms are not severe should be transported to the hospital with primary medical care available, or the hospital with advanced medical care available, whichever is nearer, with a higher priority on transporting the user to a hospital as soon as possible.

On the other hand, if a negative judgment is made in step S190, the procedure moves on to step S210. In step S210, the control device 302 judges whether or not the total value of the scores in the above-mentioned automatic triage process or question-type triage process is four or more. If an affirmative judgment is made in step S210, the procedure moves on to step S220, and the control device 302 judges whether or not the difference between the time required to reach the hospital with advanced medical care available that is the nearest to the user's current location (CSC) and the time required to reach the hospital with primary medical care available that is the nearest to the user's current location (PSC) is longer than a preset threshold T5.

In view of a situation in which the user is not taking an anticoagulant or a blood thinner, the age is younger than 80, the estimated elapsed time before arrival is longer than 270 minutes, and the total value of the scores is four or more, there is a possibility that the severity level of the symptoms of the user is high, and therefore, it is considered that it is preferable to transport the user to the hospital with advanced medical care available. However, even in such a situation, if the difference between the time required to reach the hospital with advanced medical care available that is the nearest to the user's current location (CSC) and the time required to reach the hospital with primary medical care available that is the nearest to the user's current location (PSC) is a predetermined time or longer, there is a case in which a higher priority should be placed on quick transport to a hospital without being bound by transport to a hospital with advanced medical care available. The value of the threshold T5 is set in view of such circumstances, and it is assumed in the embodiment that, for example, 60 minutes is set as the threshold T5. Note that the threshold T5 is set at an optimum value in view of the above circumstances, and is not limited to 60 minutes.

If an affirmative judgment is made in step S220, the procedure moves on to step S200, and the control device 302 determines that the user should be transported to the hospital with primary medical care available, or the hospital with advanced medical care available, in the field of the disease that the user has developed, whichever is nearer. As a result, if the total value of the scores is high and there is a high possibility that the symptoms are severe, but the difference between the time required to reach the nearest hospital with advanced medical care available (CSC) and the time required to reach the nearest hospital with primary medical care available (PSC) is longer than the threshold T5, it is possible to determine that the user should be transported to the hospital with primary medical care available, or the hospital with advanced medical care available, whichever is nearer, to place a higher priority on quickly transporting the user to a hospital.

In contrast, if a negative judgment is made in step S220, the procedure moves on to step S240, and the control device 302 determines that the user should be transported to the hospital with advanced medical care available in the field of the disease that the user has developed. As a result, if the difference between the time required to reach the nearest hospital with advanced medical care available (CSC) and the time required to reach the nearest hospital with primary medical care available (PSC) is short, it is possible to determine that the user having a high total value of the scores and a high possibility that the symptoms are severe should be transported to the hospital with advanced medical care available that can provide treatment appropriate to the severity of the symptoms of the user.

On the other hand, if a negative judgment is made in step S210, the procedure moves on to step S230, and the control device 302 judges whether or not the difference between the time required to reach the hospital with advanced medical care available that is the nearest to the user's current location (CSC) and the time required to reach the hospital with primary medical care available that is the nearest to the user's current location (PSC) is longer than a preset threshold T6.

In view of a situation in which the user is not taking an anticoagulant or a blood thinner, the age is younger than 80, the estimated elapsed time before arrival is longer than 270 minutes, and the total value of the scores is equal to or greater than two and less than four, there is a possibility that the severity level of the symptoms of the user is high, and therefore, it is considered that it is preferable to transport the user to the hospital with advanced medical care available. However, even in such a situation, if the difference between the time required to reach the hospital with advanced medical care available that is the nearest to the user's current location (CSC) and the time required to reach the hospital with primary medical care available that is the nearest to the user's current location (PSC) is a predetermined time or longer, there is a case in which a higher priority should be placed on quick transport to a hospital without being bound by transport to a hospital with advanced medical care available. The value of the threshold T6 is set in view of such circumstances, and it is assumed in the embodiment that, for example, 30 minutes is set as the threshold T6. Note that the threshold T6 is set at an optimum value in view of the above circumstances, and is not limited to 30 minutes.

If an affirmative judgment is made in step S230, the procedure moves on to step S200, and the control device 302 determines that the user should be transported to the hospital with primary medical care available, or the hospital with advanced medical care available, in the field of the disease that the user has developed, whichever is nearer. As a result, if the total value of the scores is high and there is a high possibility that the symptoms are severe, but the difference between the time required to reach the nearest hospital with advanced medical care available (CSC) and the time required to reach the nearest hospital with primary medical care available (PSC) is longer than the threshold T6, it is possible to determine that the user should be transported to the hospital with primary medical care available, or the hospital with advanced medical care available, whichever is nearer, to place a higher priority on quickly transporting the user to a hospital.

In contrast, if a negative judgment is made in step S230, the procedure moves on to step S240, and the control device 302 determines that the user should be transported to the hospital with advanced medical care available in the field of the disease that the user has developed. As a result, if the difference between the time required to reach the nearest hospital with advanced medical care available (CSC) and the time required to reach the nearest hospital with primary medical care available (PSC) is short, it is possible to determine that the user having a high total value of the scores and a high possibility that the symptoms are severe should be transported to the hospital with advanced medical care available that can provide treatment appropriate to the severity of the symptoms of the user.

If a negative judgment is made in step S180, the procedure moves on to step S250, and the control device 302 judges whether or not the total value of the scores in the above-mentioned automatic triage process or question-type triage process is less than four. Note that it is assumed that if, in step S250, in terms of a user aged younger than 80 who is not taking an anticoagulant or a blood thinner, the estimated elapsed time before arrival is equal to or shorter than the allowable time before administration of a thrombolytic agent (t-PA), it is possible to determine that the severity level of the symptoms of the user is low in a case where the total value of the scores in the automatic triage process or in the question-type triage process is less than four, and it is possible to determine that the severity level of the symptoms of the user is high in a case where the total value of the scores is four or more.

If an affirmative judgment is made in step S250, the procedure moves on to step S260, and the control device 302 determines that the user should be transported to the hospital with primary medical care available, or the hospital with advanced medical care available, in the field of the disease that the user has developed, whichever is nearer. As a result, it is possible to determine that the user having a high possibility that the symptoms are not severe should be transported to the hospital with primary medical care available, or the hospital with advanced medical care available, whichever is nearer, with a higher priority on transporting the user to a hospital as soon as possible.

On the other hand, if a negative judgment is made in step S250, the procedure moves on to step S270. In step S270, the control device 302 judges whether or not the difference between the time required to reach the hospital with advanced medical care available that is the nearest to the user's current location (CSC) and the time required to reach the hospital with primary medical care available that is the nearest to the user's current location (PSC) is longer than a preset threshold T7.

In view of a situation in which the user is not taking an anticoagulant or a blood thinner, the age is younger than 80, the estimated elapsed time before arrival is 270 minutes or less, and the total value of the scores is four or more, there is a possibility that the severity level of the symptoms of the user is high, and therefore, it is considered that it is preferable to transport the user to the hospital with advanced medical care available. However, even in such a situation, if the difference between the time required to reach the hospital with advanced medical care available that is the nearest to the user's current location (CSC) and the time required to reach the hospital with primary medical care available that is the nearest to the user's current location (PSC) is a predetermined time or longer, there is a case in which a higher priority should be placed on quick transport to a hospital without being bound by transport to a hospital with advanced medical care available. The value of the threshold T7 is set in view of such circumstances, and it is assumed in the embodiment that, for example, 30 minutes is set as the threshold T7. Note that the threshold T7 is set at an optimum value in view of the above circumstances, and is not limited to 30 minutes.

If an affirmative judgment is made in step S270, the procedure moves on to step S260, and the control device 302 determines that the user should be transported to the hospital with primary medical care available, or the hospital with advanced medical care available, in the field of the disease that the user has developed, whichever is nearer. As a result, if the total value of the scores is high and there is a high possibility that the symptoms are severe, but the difference between the time required to reach the nearest hospital with advanced medical care available (CSC) and the time required to reach the nearest hospital with primary medical care available (PSC) is longer than the threshold T7, it is possible to determine that the user should be transported to the hospital with primary medical care available, or the hospital with advanced medical care available, whichever is nearer, to place a higher priority on quickly transporting the user to a hospital.

In contrast, if a negative judgment is made in step S270, the procedure moves on to step S280, and the control device 302 determines that the user should be transported to the hospital with advanced medical care available in the field of the disease that the user has developed. As a result, if the difference between the time required to reach the nearest hospital with advanced medical care available (CSC) and the time required to reach the nearest hospital with primary medical care available (PSC) is short, it is possible to determine that the user having a high total value of the scores and a high possibility that the symptoms are severe should be transported to the hospital with advanced medical care available that can provide treatment appropriate to the severity of the symptoms of the user.

Next, the triage process for infectious disease determination is described which is executed when it has been determined that there is a possibility that the user has developed an infectious disease. In the embodiment, the triage process for infectious disease determination is an automatic triage process in which the control device 302 determines the severity of symptoms of the user on the basis of the user's healthcare information and vital data, and decides a transport destination hospital.

The control device 302 reads the user's healthcare information from the storage medium 303 on the basis of the user ID included in the measurement data received from user terminal 200. The control device 302 then executes the automatic triage process on the basis of the healthcare information that has been read out and the vital data received from the user terminal 200, and determines which hospital the user should be transported to, a hospital with advanced medical care available, a hospital with secondary medical care available, or a hospital with primary medical care available.

In the automatic triage process in the triage process for infectious disease determination, the control device 302 converts determination results of the following determination items (C1) to (C12) into numbers, and calculates a total value of the numbers as an index value indicating the severity of the symptoms of the user. In the embodiment, an example is described in which scores corresponding to the determination results are assigned to the following determination items (C1) to (C12), and the total value of the scores for the determination items (C1) to (C12) is calculated as the index value indicating the severity of the symptoms of the user in the triage for infectious disease determination in the automatic triage process. Note that the scores are set in such a manner that the total value increases with increasing possibility of becoming severe when the user develops an infectious disease; therefore, a higher total value indicates a higher possibility that the severity level of the symptoms of the user is high.
(C1) If the age of the user is 60 or older, the score is +1 and, if the age of the user is younger than 60, the score is −1.
(C2) If the user's past medical history includes cancer, the score is +2 and, if the user's past medical history does not include cancer, the score is −1.
(C3) If the user's past medical history includes a respiratory disease, the score is +2 and, if the user's past medical history does not include a respiratory disease, the score is −1.
(C4) If the user's past medical history includes a circulatory disease, the score is +2 and, if the user's past medical history does not include a circulatory disease, the score is −1.
(C5) On the basis of the data indicating the user's health condition, if the user has malaise, the score is +1 and, if the user does not have malaise, the score is −1.
(C6) On the basis of the data indicating the user's health condition, if the user has breathlessness, the score is +1 and, if the user does not have breathlessness, the score is −1.
(C7) On the basis of the data indicating the user's health condition, if the user has diarrhea and/or vomiting, the score is +1 and, if the user has diarrhea and/or vomiting, the score is −1.
(C8) If the user's body temperature is 38.5 degrees or more when an abnormality in the user is detected, the score is +1 and, if the user's body temperature is less than 38.5 degrees, the score is −1.
(C9) If the user's oxygen saturation level is less than 93 when the abnormality in the user is detected, the score is +2 and, if the user's oxygen saturation level is 93 or more, the score is −2.
(C10) On the basis of the data indicating the user's health condition, if 10 days have not passed since the user was vaccinated, the score is +1 and, if 10 days or more have passed, the score is −1.
(C11) On the basis of the data indicating the user's health condition, if the user had close contact with an infected person within five days, the score is +2 and, if the user did not have close contact with an infected person within five days, the score is −2.
(C12) On the basis of the data indicating the user's health condition, if pneumonia can be seen in a chest CT image, the score is +3 and if pneumonia cannot be seen in the chest CT image, the score is −1.

The control device 302 executes a process in a flow illustrated in FIG. 6 and, consequently specifies a transport destination appropriate to the severity of the symptoms of the user on the basis of the total value of the scores based on the determination items (C1) to (C12) calculated in the above process. The process illustrated in FIG. 6 is described below.

In step S291, the control device 302 judges whether or not the total value of the scores in the above-mentioned automatic triage process is less than five. If an affirmative judgment is made in step S291, the procedure moves on to step S295, and the control device 302 determines that the user should be transported to a hospital with primary medical care available for infectious diseases. As a result, it is possible to determine that the severity level of the symptoms of the user having a high possibility that the symptoms are not severe is low, and determine that the user should be transported to the hospital with primary medical care available for infectious diseases.

In contrast, if a negative judgment is made in step S291, the procedure moves on to step S292. In step S292, the control device 302 judges whether or not the user's past medical history includes a respiratory disease, whether or not the numerical value of the oxygen saturation data included in the vital data is less than a predetermined value, for example, 93, or whether or not pneumonia can be seen in a chest CT image of the user. If an affirmative judgment is made in step S292, the procedure moves on to step S293.

In step S293, the control device 302 determines that the user should be transported to a hospital with advanced medical care available for infectious diseases. As a result, users having a possibility of having severe symptoms, such as users having a respiratory disease in their past medical history, users with a low oxygen saturation level, and users having a possibility of having developed pneumonia, can be determined to have a high severity level of the symptoms, and it is possible to determine that the users should be transported to hospitals with advanced medical care available for infectious diseases.

In contrast, if a negative judgment is made in step S292, the procedure moves on to step S294. In step S294, the control device 302 determines that the user should be transported to a hospital with secondary medical care available for infectious diseases. As a result, if the result of the automatic triage process indicates that there is a possibility that the severity level of the symptoms is high, but the respiratory condition shows that there is a possibility that the severity level of the symptoms is intermediate, it is possible to determine that the user should be transported to a hospital with secondary medical care available for infectious diseases, or a hospital with secondary medical care available for infectious diseases, whichever is nearer, considering that there is a possibility that the hospital with secondary medical care available for infectious diseases can also provide care.

The control device 302 decides a transport destination hospital for the user on the basis of the result obtained by executing the process illustrated in FIG. 5 or 6. Specifically, if, as a result of the process illustrated in FIG. 5, the control device 302 determines that the user should be transported to a hospital with advanced medical care available that can provide care in a field of a disease that the user has developed, the control device 302 decides, as the transport destination hospital for the user, a hospital with advanced medical care available that can provide advanced medical care in the field of the disease that the user has developed, the hospital being located nearest to the user's current location, on the basis of the hospital information recorded in the storage medium 303. On the other hand, if, as a result of the process illustrated in FIG. 5, the control device 302 determines that the user should be transported to a hospital with primary medical care available, or a hospital with advanced medical care available, that can provide care in the field of the disease that the user has developed, whichever is nearer, the control device 302 decides, as the transport destination hospital for the user, a hospital located nearest to the user's current location among hospitals with advanced and primary medical care available that can provide care in the field of the disease that the user has developed, on the basis of the hospital information recorded in the storage medium 303.

Moreover, if, as a result of the process illustrated in FIG. 6, the control device 302 determines that the user should be transported to a hospital with advanced medical care available for infectious diseases, the control device 302 decides, as the transport destination hospital for the user, a hospital with advanced medical care available that can provide advanced medical care for infectious diseases, the hospital being located nearest to the user's current location, on the basis of the hospital information recorded in the storage medium 303. If, as a result of the process illustrated in FIG. 6, the control device 302 determines that the user should be transported to a hospital with secondary medical care available for infectious diseases, the control device 302 decides, as the transport destination hospital for the user, a hospital with secondary medical care available that can provide secondary medical care for infectious diseases, the hospital being located nearest to the user's current location, on the basis of the hospital information recorded in the storage medium 303. If, as a result of the process illustrated in FIG. 6, the control device 302 determines that the user should be transported to a hospital with primary medical care available for infectious diseases, the control device 302 decides, as the transport destination hospital for the user, a hospital with primary medical care available that can provide primary medical care for infectious diseases, the hospital being located nearest to the user's current location, on the basis of the hospital information recorded in the storage medium 303.

After deciding the transport destination hospital for the user, the control device 302 transmits information for identifying the decided transport destination hospital to the user terminal 200. Examples of the information for identifying the transport destination hospital that is transmitted to the user terminal 200 include information such as the hospital's name, location information, and telephone number.

In the user terminal 200, after receiving the information for identifying the transport destination hospital from the monitoring apparatus 300, the control device 204 outputs the received information to the touchscreen 201 to display the information thereon. As a result, a person who transports the user to a hospital, such as an emergency medical technician, can check the information displayed on the touchscreen 201 of the user terminal 200 and find the hospital where the user should be transported.

After deciding the transport destination hospital for the user, the control device 302 identifies an email address of the transport destination hospital on the basis of the hospital information recorded in the storage medium 303, and transmits person-to-be-transported information about the user to be transported, to the identified email address. The person-to-be-transported information includes the above-mentioned healthcare information, vital data, and data indicating determination results of the determination items (A1) to (A10), data indicating response contents to the question items (B1) to (B12), or data indicating determination results of the determination items (C1) to (C12). As a result, the transport destination hospital can grasp in advance the healthcare information, vital data, and data indicating the triage result of the patient to be accepted. The transport destination hospital can be ready in advance for acceptance appropriate to the symptoms of the user, and the user can receive appropriate treatment quickly at the transport destination, so that the survival rate of the user can be increased.

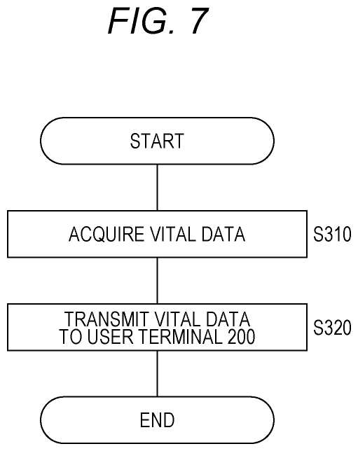

FIG. 7 is a flowchart illustrating the flow of a process that is executed by the vital data measuring apparatus 100 in the embodiment. As described above, the process illustrated in FIG. 7 is executed by the control device 103 as a program that is started at a preset vital data transmission timing.

In step S310, the control device 103 acquires the user's vital data inputted through the vital data measuring unit 102. The procedure then moves on to step S320.

In step S320, the control device 103 transmits the vital data acquired in step S310 to the user terminal 200 through the communication module 101. The process then ends.

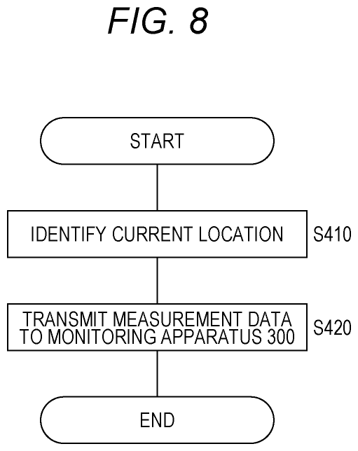

FIG. 8 is a flowchart illustrating the flow of a measurement data transmission process that is executed by the user terminal 200 in the embodiment. The process illustrated in FIG. 8 is executed by the control device 204 as a program that is started when the user's vital data is received from the vital data measuring apparatus 100.

In step S410, the control device 204 identifies the current location of the user terminal 200 by acquiring the above-mentioned current location information on the basis of the output from the GPS module 203. The procedure then moves on to step S420.

In step S420, the control device 204 transmits measurement data including the vital data received from the vital data measuring apparatus 100, the information for identifying the user, for example, the above-mentioned user ID, and the current location information indicating the current location of the user terminal 200 identified in step S410, to the monitoring apparatus 300 through the communication module 202. The process then ends.

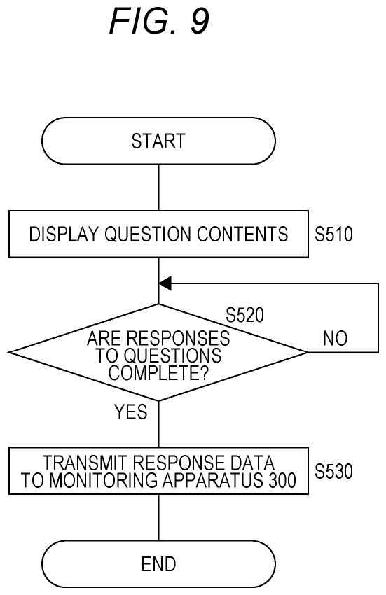

FIG. 9 is a flowchart illustrating the flow of a response data transmission process that is executed by the user terminal 200 in the embodiment. As described above, the process illustrated in FIG. 9 is executed by the control device 204 as a program that is started when the monitoring apparatus 300 gives an instruction to output the questions for the question-type triage process.

When in step S510 the monitoring apparatus 300 instructs the control device 204 to output the questions, the control device 204 outputs the question contents corresponding to the above-mentioned question items (B1) to (B12) to the touchscreen 201 and displays the question contents thereon. The procedure then moves on to step S520.

In step S520, the control device 204 judges whether or not acceptance of the input of a response to each of the question items has been completed. For example, the screen is configured in such a manner that an operator touches a send button after inputting responses to the question items, and the control device 204 is simply required to make a judgment in step S520 by checking whether or not an input has been performed for the each of the question items when having detected that the send button has been touched. Note that in terms of question items for which the question-type triage process can be performed as long as a response to one of two questions can be obtained as in (B3) and (B4), it is assumed that it is simply required to input a response to only the one of the two questions. If an affirmative judgment is made in step S520, the procedure moves on to step S530.

In step S530, the control device 204 transmits, to the monitoring apparatus 300, response data indicating response contents of which inputs have been accepted on the touchscreen 201. The process then ends.

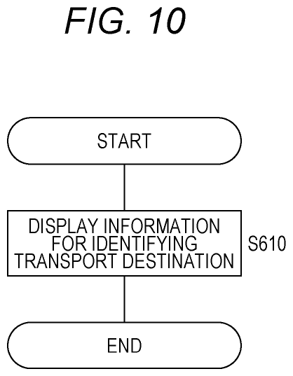

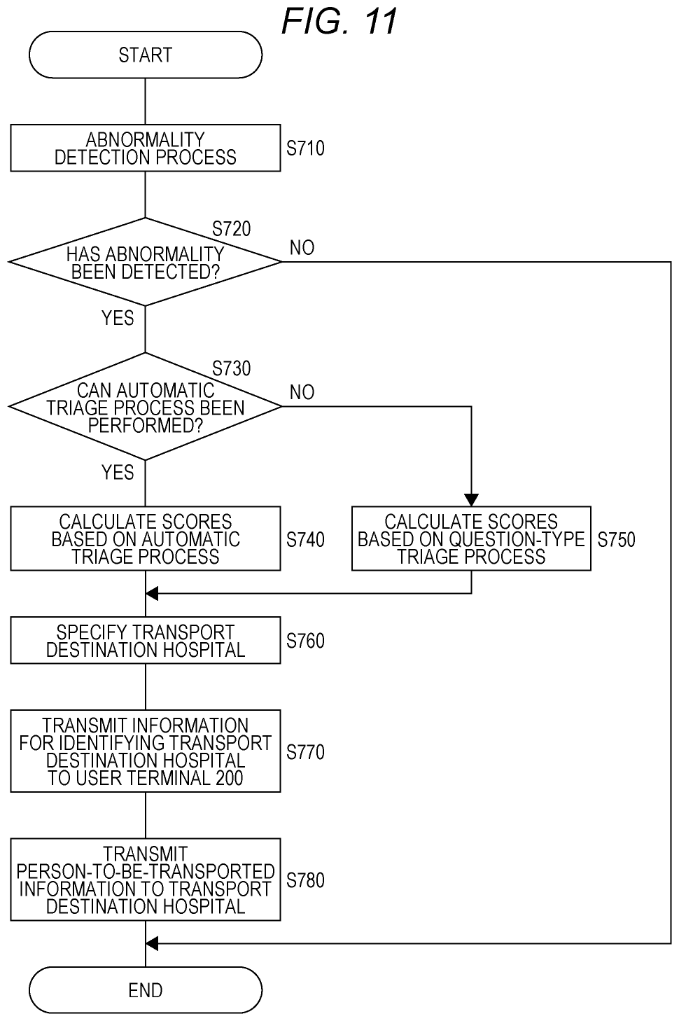

FIG. 10 is a flowchart illustrating the flow of a transport destination hospital information output process that is executed by the user terminal 200 in the embodiment. The process illustrated in FIG. 10 is executed by the control device 204 as a program that is started when information for identifying the transport destination hospital for the user is received from the monitoring apparatus 300.

In step S610, the control device 204 outputs the information for identifying the transport destination hospital received from the monitoring apparatus 300 to the touchscreen 201 and displays the information thereon. The process then ends.

FIG. 11 is a flowchart illustrating the flow of a process that is executed by the monitoring apparatus 300 in the embodiment. The process illustrated in FIG. 11 is executed by the control device 302 as a program that is started when the above-mentioned measurement data is received from the user terminal 200. Note that, in FIG. 11, it is assumed that data necessary for the above-mentioned registration of the user information and processing of, for example, data of the hospital information is prerecorded in the storage medium 303.

In step S710, as described above, the control device 302 detects an abnormality in the user by determining whether or not there is a possibility that the user has developed heart disease, brain disease, or an infectious disease, on the basis of the user's vital data included in the measurement data received from the user terminal 200. The procedure then moves on to step S720.

In step S720, the control device 302 judges whether or not an abnormality in the user has been detected as a result of the abnormality detection process in step S710. If a negative judgment is made in step S720, the process ends. In contrast, if an affirmative judgment is made in step S720, the procedure moves on to step S730.

In step S730, the control device 302 judges whether or not the above-mentioned automatic triage process can be performed. In other words, as described above, if it is determined that determinations can be made on the determination items (A1) to (A10) on the basis of the user's healthcare information and vital data in the triage process for disease determination, or in the case of the triage process for infectious disease determination, the control device 302 makes an affirmative judgment in step S730. On the other hand, if, in the triage process for disease determination, the healthcare information and the information on the vital data are insufficient and it is determined that a determination cannot be made on any of the determination items (A1) to (A10), the control device 302 makes a negative judgment in step S730.

If an affirmative judgment is made in step S730, the procedure moves on to step S740, and, as described above, the control device 302 coverts determination results of the determination items (A1) to (A10) or (C1) to (C12) into numbers, and calculates the total value of the numbers as the score for determining a transport destination appropriate to the severity of the symptoms of the user. The procedure then moves on to step S760 described below.

In contrast, if a negative judgment is made in step S730, the procedure moves on to step S750, and, as described above, the control device 302 obtains response data to the question items (B1) to (B12) from the user terminal 200, converts the response results into numbers, and calculates the total value of the numbers as the score for determining a transport destination appropriate to the severity of the symptoms of the user. The procedure then moves on to step S760.

In step S760, the control device 302 executes the above-mentioned process illustrated in FIG. 5 or 6 on the basis of the total value of the scores in the automatic triage process calculated in step S740, or the total value of the scores in the question-type triage process calculated in step S750, and specifies a transport destination hospital for the symptoms of the user on the basis of the execution result. The procedure then moves on to step S770.

In step S770, as described above, the control device 302 transmits information for identifying the decided transport destination hospital, to the user terminal 200. The procedure then moves on to step S780.

In step S780, as described above, the control device 302 identifies an email address of the transport destination hospital on the basis of the hospital information recorded in the storage medium 303, and transmits person-to-be-transported information about the user to be transported to the identified email address. The process then ends.

According to the embodiment described above, the following operations and effects can be obtained:
(1) In the user terminal 200 held by a user who is a subject, the control device 204 acquires vital data including the user's blood pressure data, pulse data, electrocardiogram data, and oxygen saturation data from the vital data measuring apparatus 100, and transmits the acquired vital data to the monitoring apparatus 300. The monitoring apparatus 300 is configured in such a manner that when having determined that there is a possibility that the subject has developed heart disease, brain disease, or an infectious disease on the basis of the vital data received from the user terminal 200, the control device 302 detects an abnormality in the user and that when having detected the abnormality in the user, the control device 302 executes the above-mentioned triage process, specifies a transport destination hospital to which the user is transported on the basis of the severity of symptoms of the user, and transmits the specification result to the user terminal 200. As a result, when there is a possibility that the user has developed heart disease, brain disease, or an infectious disease, it is possible to execute the triage process, determine the severity of the symptoms of the user, and specify a transport destination hospital appropriate to the severity of the symptoms. In this manner, when the subject has developed heart disease, brain disease, or an infectious disease, a transport destination hospital appropriate to the severity of symptoms of the user is specified quickly, and therefore, it is possible to promptly transport the user to an appropriate hospital, and consequently, it is possible to increase the survival rate of the user.
(2) The control device 302 is configured in such a manner as to determine the severity of the symptoms of the user on the basis of healthcare information recorded in the storage medium 303 for the user's healthcare, and the vital data. As a result, it is possible to execute the automatic triage process on the basis of the user's healthcare information and vital data and to quickly determine the severity of the symptoms of the user.
(3) The control device 302 is configured in such a manner as to convert a determination result into a number for each of the preset determination items (A1) to (A10) or (C1) to (C12) on the basis of the healthcare information and the vital data, calculate the total value of the numbers identified from the determination items as the index value, and consequently determine the severity of the symptoms of the user. As a result, if the determination items for determining the severity of the symptoms of the user and the scores corresponding to the determination results of the determination items are preset, the index value indicating the severity of the symptoms of the user can be calculated with high accuracy.
(4) The control device 302 is configured in such a manner as to, if, in the triage process for disease determination, the severity of the symptoms of the user cannot be determined by the automatic triage process based on the healthcare information and the vital data, acquire response data to the questions for checking the symptoms of the user from the user terminal 200, and determine the severity of the symptoms of the user on the basis of the acquired response data. As a result, even if the automatic triage process cannot be executed, it is possible to determine the severity of the symptoms based on the actual condition of the user recognized by an emergency medical technician or a family member, on the basis of the response contents to the questions for determining the symptoms of the user.
(5) The control device 302 is configured in such a manner as to convert a response result into a number for each of the plurality of preset question items on the basis of the response data, calculate the total value of the numbers identified from the question items as the index value, and consequently determine the severity of the symptoms of the user. As a result, if the question items for determining the severity of the symptoms of the user and the scores corresponding to the response contents to the question items are preset, the index value indicating the severity of the symptoms of the user can be calculated with high accuracy.
(6) The control device 302 is configured in such a manner as to transmit, to the email address of the specified transport destination hospital, the person-to-be-transported information including the healthcare information, the vital data, and the data indicating the severity of the symptoms of the user, that is, the data indicating the determination results of the determination items (A1) to (A10), the data indicating the response contents to the question items (B1) to (B12), or the data indicating the determination results of the determination items (C1) to (C12). As a result, the transport destination hospital can grasp in advance the healthcare information, vital data, and data indicating the triage result of the patient to be accepted.
(7) The control device 302 is configured in such a manner as to, if the control device 302 determines that the severity level of the symptoms of brain disease or heart disease of the user is low, select a hospital nearer to the user's current location from a hospital with primary medical care available in the field of the disease matching the symptoms of the user and a hospital with advanced medical care available in the field of the disease matching the symptoms of the user, and specify the hospital as the transport destination hospital. As a result, in order to increase the survival rate, a transport destination hospital can be specified for a user having a high possibility that the symptoms are not severe, placing a higher priority on prompt transport to a hospital.
(8) The control device 302 is configured in such a manner as to, if the control device 302 determines that the severity level of the symptoms of brain disease or heart disease of the user is high, when the difference between the time required to reach a hospital with advanced medical care available that is the nearest to the user's current location and the time required to reach a hospital with primary medical care available that is the nearest to the user's current location is longer than a preset threshold, select a hospital nearer to the user's current location from the hospital with primary medical care available and the hospital with advanced medical care available, and specify the hospital as the transport destination hospital. Moreover, it is configured in such a manner that when the difference between the time required to reach the hospital with advanced medical care available that is the nearest to the user's current location and the time required to reach the hospital with primary medical care available that is the nearest to the user's current location is equal to or shorter than the preset threshold, a hospital that is near to the user's current location is selected from the hospital with advanced medical care available and is specified as the transport destination hospital. As a result, if there is a high possibility that the symptoms of the user are severe, but the difference between the time required to reach the nearest hospital with advanced medical care available and the time required to reach the nearest hospital with primary medical care available is longer than the predetermined threshold, it is possible to determine that the user should be transported to the hospital with primary medical care available, or the hospital with advanced medical care available, whichever is nearer, to place a higher priority on quick transport to a hospital. On the other hand, if the difference between the time required to reach the nearest hospital with advanced medical care available and the time required to reach the nearest hospital with primary medical care available is short, it is possible to determine that the user should be transported to the hospital with advanced medical care available to allow the user to receive sophisticated treatment appropriate to the severity of the symptoms of the user.
(9) The control device 302 is configured in such a manner as to determine that there is a possibility that the user has developed heart disease when the waveform shown in the electrocardiogram data included in the user's vital data is a preregistered characteristic waveform of heart disease, determine that there is a possibility that the user has developed brain disease when the numerical value shown in the blood pressure data and the waveform shown in the pulse data are a preregistered characteristic numerical value of and a preregistered characteristic waveform of brain disease, and determine that there is a possibility that the user has developed an infectious disease when the numerical value of the user's oxygen saturation data is a numerical value suspected of having developed an infectious disease, or if pneumonia can be seen in a chest CT image of the user. As a result, an abnormality in the user can be detected with high accuracy on the basis of the user's vital data.
(10) The user terminal 200 is configured in such a manner that the control device 204 acquires the viral data from the vital data measuring apparatus 100 that is a wearable terminal the user wears. This makes it possible to regularly acquire the user's vital data.

### MODIFICATIONS

Note that the medical support system of the above-mentioned embodiment can also be modified as follows:
(1) In the above-mentioned embodiment, the example is described in which if the control device 302 determines that the user should be transported to a hospital with advanced medical care available that can provide care in a field of a disease that the user has developed, the control device 302 decides, as a transport destination hospital for the user, a hospital with advanced medical care available that can provide care in the field of the disease that the user has developed, the hospital being located nearest to the user's current location, on the basis of the hospital information recorded in the storage medium 303. Moreover, the example is described in which if the control device 302 determines that the user should be transported to a hospital with primary medical care available, or a hospital with advanced medical care available, which can provide care in the field of the disease that the user has developed, whichever is nearer, the control device 302 decides, as a transport destination hospital for the user, a hospital that is located nearer to the user's current location from a hospital with advanced medical care available and a hospital with primary medical care, which can provide care in the field of the disease that the user has developed, on the basis of the hospital information recorded in the storage medium 303. However, upon deciding a transport destination hospital to which the user is transported, the control device 302 may be configured in such a manner as to also determine whether or not there is a specialist related to the field of the disease that the user has developed, whether or not a care unit for the field of the disease that the user has developed is available, and whether or not the current time is within the emergency medical response hours, on the basis of the hospital information recorded in the storage medium 303, and prioritize and select a hospital having a specialist, a hospital having an available care unit, and a hospital that can respond immediately.

Moreover, the control device 302 may be configured in such a manner as to select a plurality of hospitals in ascending order of distance to the user's current location, or select hospitals including a hospital with a specialist or a hospital with an available care unit, present a plurality of candidates for a hospital to be a transport destination for the user, and specify a hospital that is designated by an emergency medical technician on the user terminal 200, as the transport destination from among the plurality of candidates. As a result, it possible to present options of the transport destination to the emergency medical technician. Moreover, even if the candidates include a hospital that cannot accept the patient, the emergency medical technician can still select a transport destination hospital from among the other candidates.
(2) In the above-mentioned embodiment, the example is described in which the control device 302 executes the triage process and decides a transport destination hospital for the user if an abnormality in the user is detected. In addition, the control device 302 may be configured in such a manner as to send notification that an abnormality in the user has been detected, to a preregistered notification destination. For example, it is configured in such a manner that the contact information of a person concerned such as the user's family doctor or family member can be included and registered as a notification destination in the healthcare information and, if a notification destination is registered in the user's healthcare information, the control device 302 is simply required to transmit, to the notification destination, information indicating that the abnormality in the user has been detected. As a result, the person concerned such as a family doctor or family member can grasp the occurrence of the abnormality in the user. Note that, if an email address is registered as the contact information, information may be transmitted to the email address and, if a mobile phone number is registered, a push notification that uses a short message service (SMS) may be sent to the mobile phone number.

Moreover, the control device 302 may be configured in such a manner as to, if the control device 302 detects an abnormality in the user, send notification of the occurrence of the abnormality in the user together with the user's current location, to a preregistered emergency notification destination, for example, a fire center. As a result, when an abnormality is detected in the user, an emergency call can be made quickly. Moreover, an emergency medical technician who has rushed to the scene in response to the call operates the user terminal 200 and can check a transport destination hospital specified by the automatic triage process, and respond to the questions for the question-type triage process.
(3) In the above-mentioned embodiment, the example is described in which when the numerical value of the oxygen saturation data included in the vital data is the numerical value that is preregistered in the storage medium 303 and is suspected of an infectious disease, or when pneumonia can be seen in a chest CT image of the user, the control device 302 determines that there is a possibility that the user has developed an infectious disease, and detects an abnormality in the user. However, it may be configured in such a manner that when it is detected that the waveforms shown in the electrocardiogram data and pulse data included in the vital data is the characteristic waveforms of an infectious disease preregistered in the storage medium 303, it is determined that there is a possibility that the user has developed the infectious disease, and an abnormality in the user is detected.
(4) In the above-mentioned embodiment, the example is described in which the vital data including the user's blood pressure data, pulse data, electrocardiogram data, body temperature data, and oxygen saturation data is acquired by the vital data measuring apparatus 100 and inputted into the user terminal 200. However, in terms of the user's blood pressure data, pulse data, electrocardiogram data, body temperature data, and oxygen saturation data, it may be configured in such a manner that the user inputs, on the user terminal 200, data measured using, for example, a sphygmomanometer, an electrocardiograma pulse oximeter, and a clinical thermometer.
(5) In the above-mentioned embodiment, the example is described in which the vital data measuring apparatus 100 acquires the blood pressure data, the pulse data, the electrocardiogram data, the body temperature data, and the oxygen saturation data. However, it may be configured in such a manner that if one vital data measuring apparatus 100 can acquire only part of these pieces of data, the user wears a plurality of the vital data measuring apparatuses 100 to acquire the blood pressure data, the pulse data, the electrocardiogram data, the body temperature data, and the oxygen saturation data. Moreover, it may be configured in such a manner that if the vital data measuring apparatus 100 can acquire only part of these pieces of data, only acquirable data is acquired using the vital data measuring apparatus 100, and the user inputs, on the user terminal 200, the other pieces of data measured using, for example, a sphygmomanometer, an electrocardiograma pulse oximeter, and a clinical thermometer.

Note that the present invention is not limited to the configuration in the above-mentioned embodiment at all as long as the characteristic functions of the present invention are not impaired. Moreover, the configuration may be a combination of the above-mentioned embodiment and a plurality of modifications thereof.

### LIST OF REFERENCE SIGNS

10 Medical support system
100 Vital data measuring apparatus
101 Communication module
102 Vital data measuring unit
103 Control device
200 User terminal
201 Touchscreen
202 Communication module
203 GPS module
204 Control device
300 Monitoring apparatus
301 Connection interface
302 Control device
303 Storage medium

1. A medical support system comprising:
a vital data acquisition means configured to acquire vital data including blood pressure data, pulse data, electrocardiogram data, and oxygen saturation data of a subject;
an abnormality detection means configured to, upon determining that there is a possibility that the subject has developed heart disease, brain disease, or an infectious disease on the basis of the vital data acquired by the vital data acquisition means, detect the possibility that the subject has developed heart disease, brain disease, or an infectious disease, as an abnormality in the subject;
a determination means configured to, upon the abnormality detection means detecting the abnormality, determine the severity of symptoms of the subject;
a transport destination specification means configured to specify a transport destination hospital to which the subject is transported, on the basis of the severity of the symptoms of the subject determined by the determination means; and
a transport destination information output means configured to output transport destination information about the transport destination hospital specified by the transport destination specification means.

2. The medical support system according to claim 1, wherein
the determination means determines the severity of the symptoms of the subject on the basis of healthcare information recorded in a storage medium for healthcare of the subject, and the vital data.

3. The medical support system according to claim 2, wherein
the determination means converts a determination result into a number for each of a plurality of preset determination items on the basis of the healthcare information and the vital data, calculates a total value of the numbers identified from the determination items, as an index value indicating the severity of the symptoms of the subject, and consequently determines the severity of the symptoms of the subject.

4. The medical support system according to claim 2, further comprising:
a question output means configured to output questions for checking the symptoms of the subject in a case where it is impossible to determine the severity of the symptoms of the subject on the basis of the healthcare information and the vital data; and
a response data acquisition means configured to accept responses to the questions and acquire response data, wherein
the determination means determines the severity of the symptoms of the subject on the basis of the response data acquired by the response data acquisition means.

5. The medical support system according to claim 4, wherein
the determination means converts a response result into a number for each of a plurality of preset question items on the basis of the response data, calculates a total value of the numbers identified from the question items, as an index value indicating the severity of the symptoms of the subject, and consequently determines the severity of the symptoms of the subject.

6. The medical support system according to claim 1, further comprising a person-to-be-transported information transmission means configured to transmit person-to-be-transported information including the healthcare information, the vital data, and data indicating the severity of the symptoms of the subject to the transport destination hospital specified by the transport destination specification means.

7. The medical support system according to claim 1, further comprising a notification means configured to, upon the abnormality detection means detecting the abnormality, send notification that the abnormality has been detected in the subject, to a preregistered notification destination.

8. The medical support system according to claim 1, further comprising a current location identification means configured to identify a current location of the subject, wherein
in a case where the determination means determines that the severity level of the symptoms of the subject is low, the transport destination specification means selects a hospital nearer to the current location of the subject identified by the current location identification means from a hospital with primary medical care available in a field of a disease matching the symptoms of the subject and a hospital with advanced medical care available in the field of the disease matching the symptoms of the subject, and specifies the hospital as the transport destination hospital.

9. The medical support system according to claim 8, wherein
in a case where the determination means determines that the severity level of the symptoms of the subject is high, the transport destination specification means,
upon a difference between time required to reach a hospital with advanced medical care available that is the nearest to the current location of the subject and time required to reach a hospital with primary medical care available that is the nearest to the current location of the subject being longer than a preset threshold, selects a hospital nearer to the current location of the subject identified by the current location identification means from the hospital with primary medical care available and the hospital with advanced medical care available, and specifies the hospital as the transport destination hospital, and
upon the difference between the time required to reach the hospital with advanced medical care available that is the nearest to the current location of the subject and the time required to reach the hospital with primary medical care available that is the nearest to the current location of the subject being equal to or shorter than the preset threshold, selects a hospital near to the current location of the subject identified by the current location identification means from the hospital with advanced medical care available, and specifies the hospital as the transport destination hospital.

10. The medical support system according to claim 1, wherein the abnormality detection means
determines that there is a possibility that the subject has developed heart disease upon a waveform shown in the electrocardiogram data being a preregistered characteristic waveform of heart disease,
determines that there is a possibility that the subject has developed brain disease upon a numerical value shown in the blood pressure data and a waveform shown in the pulse data being a preregistered characteristic numerical value of and a preregistered characteristic waveform of brain disease, and
determines that there is a possibility that the subject has developed an infectious disease upon a numerical value of the oxygen saturation data being a numerical value suspected of having developed an infectious disease, or in a case where pneumonia is seeable in a chest CT image of the subject.

11. The medical support system according to claim 1, wherein the vital data acquisition means acquires the vital data from a wearable terminal worn by the subject, the wearable terminal having a function of measuring the vital data.

12. A medical support system comprising:
a subject terminal held by a subject; and
a monitoring apparatus connected to the subject terminal via a communication line, wherein
the subject terminal includes:
a vital data acquisition means configured to acquire vital data including blood pressure data, pulse data, electrocardiogram data, and oxygen saturation data of the subject; and
a vital data transmission means configured to transmit, to the monitoring apparatus, the vital data acquired by the vital data acquisition means, and
the monitoring apparatus includes:
an abnormality detection means configured to, upon determining that there is a possibility that the subject has developed heart disease, brain disease, or an infectious disease on the basis of the vital data received from the subject terminal, detect the possibility that the subject has developed heart disease, brain disease, or an infectious disease, as an abnormality in the subject;
a determination means configured to, upon the abnormality detection means detecting the abnormality, determine the severity of symptoms of the subject;
a transport destination specification means configured to specify a transport destination hospital to which the subject is transported, on the basis of the severity of the symptoms of the subject determined by the determination means; and
a transport destination information transmission means configured to transmit, to the subject terminal, transport destination information about the transport destination hospital specified by the transport destination specification means.

13. The medical support system according to claim 12, wherein the subject terminal further includes:
a transport destination information receiving means configured to receive the transport destination information from the monitoring apparatus; and
a transport destination information display means configured to display, on a display device, the transport destination information received by the transport destination information receiving means.

14. The medical support system according to claim 12, wherein the determination means determines the severity of the symptoms of the subject on the basis of healthcare information recorded in a storage medium for healthcare of the subject, and the vital data.

15. The medical support system according to claim 14, wherein the determination means converts a determination result into a number for each of a plurality of preset determination items on the basis of the healthcare information and the vital data, calculates a total value of the numbers identified from the determination items, as an index value indicating the severity of the symptoms of the subject, and consequently determines the severity of the symptoms of the subject.

16. The medical support system according to claim 14, wherein
the subject terminal further includes:
a question output means configured to output questions for checking the symptoms of the subject in response to an instruction of the monitoring apparatus;
a response data acquisition means configured to accept responses to the questions and acquire response data; and
a response data transmission means configured to transmit, to the monitoring apparatus, the response data acquired by the response data acquisition means, and
the determination means instructs the subject terminal to output the questions and determines the severity of the symptoms of the subject on the basis of the response data received from the subject terminal in a case where it is impossible to determine the severity of the symptoms of the subject on the basis of the healthcare information and the vital data.

17. The medical support system according to claim 16, wherein the determination means converts a response result into a number for each of a plurality of preset question items on the basis of the response data, calculates a total value of the numbers identified from the question items, as an index value indicating the severity of the symptoms of the subject, and consequently determines the severity of the symptoms of the subject.

18. The medical support system according to claim 12, wherein the monitoring apparatus further includes a person-to-be-transported information transmission means configured to transmit person-to-be-transported information including the healthcare information, the vital data, and data indicating the severity of the symptoms of the subject to the transport destination hospital specified by the transport destination specification means.

19. The medical support system according to claim 12, wherein the monitoring apparatus further includes a notification means configured to, upon the abnormality detection means detecting the abnormality, send notification that the abnormality has been detected in the subject, to a preregistered notification destination.

20. The medical support system according to claim 12, wherein
the subject terminal further includes a current location identification means configured to identify a current location of the subject, and
in a case where the determination means determines that the severity level of the symptoms of the subject is low, the transport destination specification means selects a hospital nearer to the current location of the subject identified by the current location identification means from a hospital with primary medical care available in a field of a disease matching the symptoms of the subject and a hospital with advanced medical care available in the field of the disease matching the symptoms of the subject, and specifies the hospital as the transport destination hospital.

21. The medical support system according to claim 20, wherein
in a case where the determination means determines that the severity level of the symptoms of the subject is high, the transport destination specification means,
upon a difference between time required to reach a hospital with advanced medical care available that is the nearest to the current location of the subject and time required to reach a hospital with primary medical care available that is the nearest to the current location of the subject being longer than a preset threshold, selects a hospital nearer to the current location of the subject identified by the current location identification means from the hospital with primary medical care available and the hospital with advanced medical care available, and specifies the hospital as the transport destination hospital, and
upon the difference between the time required to reach the hospital with advanced medical care available that is the nearest to the current location of the subject and the time required to reach the hospital with primary medical care available that is the nearest to the current location of the subject being equal to or shorter than the preset threshold, selects a hospital near to the current location of the subject identified by the current location identification means from the hospital with advanced medical care available, and specifies the hospital as the transport destination hospital.

22. The medical support system according to claim 12, wherein the abnormality detection means
determines that there is a possibility that the subject has developed heart disease upon a waveform shown in the electrocardiogram data being a preregistered characteristic waveform of heart disease,
determines that there is a possibility that the subject has developed brain disease upon a numerical value shown in the blood pressure data and a waveform shown in the pulse data being a preregistered characteristic numerical value of and a preregistered characteristic waveform of brain disease, and
determines that there is a possibility that the subject has developed an infectious disease upon a numerical value of the oxygen saturation data being a numerical value suspected of having developed an infectious disease, or in a case where pneumonia is seeable in a chest CT image of the subject.

23. The medical support system according to claim 12, wherein the vital data acquisition means acquires the vital data from a wearable terminal worn by the subject, the wearable terminal having a function of measuring the vital data.
